(19) United States
(12) Patent Application Publication
(10) Pub. No.: US 2024/0316042 A1
(43) Pub. Date: Sep. 26, 2024

(54) METHODS OF TREATING PEDIATRIC PATIENTS WITH UPADACITINIB

(71) Applicant: AbbVie Inc., North Chicago, IL (US)

(72) Inventors: Mohamed-Eslam F. Mohamed, Gurnee, IL (US); Cheng Thiam Tan, Grayslake, IL (US); Henrique D. Teixeira, Green Oaks, IL (US); Alvina D. Chu, Los Altos, CA (US)

(21) Appl. No.: 18/612,955

(22) Filed: Mar. 21, 2024

Related U.S. Application Data
(60) Provisional application No. 63/623,994, filed on Jan. 23, 2024.
(60) Provisional application No. 63/491,665, filed on Mar. 22, 2023.

(51) Int. Cl. A61K 31/4985; A61P 17/00; A61P 19/02

(52) U.S. Cl. CPC A61K 31/4985; A61P 17/00; A61P 19/02

(57) ABSTRACT

The present disclosure is directed to methods for treating disease in pediatric patients with the JAK1 selective inhibitor upadacitinib. The diseases and disorders include idiopathic arthritis (pcJIA), systemic juvenile idiopathic arthritis (SJIA), juvenile psoriatic arthritis (JPsA), atopic dermatitis (AD), juvenile ankylosing spondylitis (JAS), juvenile non-radiographic spondyloarthritis (nr-axSpA), hidradenitis suppurativa (HS), systemic lupus erythematosus (SLE), ulcerative colitis (UC), and Crohn's disease (CD). The treatment methods generally comprise administering to a pediatric patient a therapeutically effective amount of upadacitinib as a stable liquid pharmaceutical composition or a solid dosage form, at a dose based on patient body weight.

### CROSS-REFERENCE TO RELATED APPLICATIONS

This application claims the benefit of U.S. Provisional Application No. 63/491,665 filed Mar. 22, 2023, and claims the benefit of U.S. Provisional Application No. 63/623,994, filed Jan. 23, 2024, each of which are herein incorporated by reference in their entirety.

### FIELD OF THE DISCLOSURE

The present disclosure is directed to methods for treating disease in pediatric patients with the JAK1 selective inhibitor upadacitinib.

### BACKGROUND OF THE DISCLOSURE

Upadacitinib is a JAK1 selective inhibitor approved for the treatment of rheumatoid arthritis, psoriatic arthritis, ankylosing spondylitis, non-radiographic spondyloarthritis, and ulcerative colitis in adults, and the treatment of atopic dermatitis in adults and adolescents 12 years of age and older and weighing 40 kg or more. Upadacitinib is also under investigation for use in treating adults having Crohn's disease, hidradenitis suppurativa and systemic lupus erythematosus. Upadacitinib has not been evaluated for the treatment of pediatric patients under 12 years of age or under 18 years of age and weighing less than 40 kg.

While the pharmacokinetics, safety, and tolerability of upadacitinib in adults is known, the pharmacokinetics, safety, and tolerability of upadacitinib in pediatric patients under 12 years of age or under 18 year of age and weighing less than 40 kg has not been studied. Because the above identified diseases also occur in pediatric patients, it is desirable in the art to provide safe and efficacious doses of upadacitinib in pediatric patients. In particular, it is desirable to establish weight-based dosing regimens which provide comparable plasma exposure in pediatric patients to doses determined as efficacious in adults (i.e., 15 mg and 30 mg QD). In addition, tablet formulations may be difficult for younger pediatric patients to swallow, which may result in poor patient compliance and reduced therapeutic efficacy.

### SUMMARY OF THE DISCLOSURE

The present disclosure provides methods for treating a disease or disorder in a pediatric patient in need thereof with upadacitinib. The diseases and disorders include idiopathic arthritis (pcJIA), systemic juvenile idiopathic arthritis (SJIA), juvenile psoriatic arthritis (JPsA), atopic dermatitis (AD), juvenile ankylosing spondylitis (JAS), juvenile non-radiographic spondyloarthritis (nr-axSpA), hidradenitis suppurativa (HS), systemic lupus erythematosus (SLE), ulcerative colitis (UC), and Crohn's disease (CD). The treatment methods generally comprise administering to a pediatric patient a therapeutically effective amount of upadacitinib as a stable liquid pharmaceutical composition or solid dosage form, at a dose based on patient body weight. Specifically, provided herein are methods of treating pediatric patients in need thereof with a therapeutically effective amount of upadacitinib, in an amount based on body weight category, as either a twice-daily stable liquid pharmaceutical composition or as a once-daily extended-release tablet.

In one aspect is provided a method for treating polyarticular course juvenile idiopathic arthritis (pcJIA) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprising administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In some embodiments, the pcJIA is rheumatoid factor-positive or rheumatoid factor-negative polyarticular JIA.

In some embodiments, the pediatric patient has a history of arthritis affecting at least 5 joints within the first 6 months of disease.

In some embodiments, the pediatric patient does not have a diagnosis of enthesitis-related arthritis (ERA) or juvenile psoriatic arthritis (JPSA).

In some embodiments, the pediatric patient has:
a) 5 or more active joints as defined by the presence of swollen joints not due to deformity; or
b) in the absence of swelling, joints with limitation of movement (LOM) plus pain on motion and/or tenderness with palpation, with LOM present in at least three of the active joints.

In some embodiments, the pediatric patient is receiving a stable dose of ≤20 mg/m2 of methotrexate for at least 8 weeks before the start of administration.

In some embodiments, the pediatric patient is receiving a stable dose of oral glucocorticoids no greater than 10 mg/day or 0.2 mg/kg/day, whatever is lower, for at least 1 week before the start of administration.

In some embodiments, the pediatric patient achieves one or more of: a JIA ACR pediatric 30/50/70/90/100 response, a change from baseline in JADAS10/27/71 responses, low disease activity according to JADAS-based criteria, or remission according to JADAS-based criteria. In some embodiments, the pediatric patient achieves one or more of: a JIA ACR pediatric 30/50/70/90/100 response, a change from baseline in JADAS10/27/71 responses, low disease activity according to JADAS-based criteria, or remission according to JADAS-based criteria is achieved at 8 weeks, 10 weeks, 12 weeks, 14 weeks, 16 weeks, 18 weeks, 20 weeks, 22 weeks, 24 weeks, 48 weeks, or 52 weeks after the first daily administration. In some embodiments, the pediatric patient achieves a JIA ACR pediatric 30 response at 12 weeks after the first daily administration. In some embodiments, the pediatric patient achieves a JIA ACR pediatric 50 response at 12 weeks after the first daily administration. In some embodiments, the pediatric patient achieves a JIA ACR pediatric 70 response at 12 weeks after the first daily administration. In some embodiments, the pediatric patient achieves a JIA ACR pediatric 90 response at 12 weeks after the first daily administration. In some embodiments, the pediatric patient achieves a JIA ACR pediatric 100 response at 12 weeks after the first daily administration.

In some embodiments, the pediatric patient achieves a JIA ACR pediatric 30 response at 24 weeks after the first daily administration. In some embodiments, the pediatric patient achieves a JIA ACR pediatric 50 response at 24 weeks after the first daily administration. In some embodiments, the pediatric patient achieves a JIA ACR pediatric 70 response at 24 weeks after the first daily administration. In some embodiments, the pediatric patient achieves a JIA ACR pediatric 90 response at 24 weeks after the first daily administration. In some embodiments, the pediatric patient achieves a JIA ACR pediatric 100 response at 24 weeks after the first daily administration.

In some embodiments, the pediatric patient achieves a JIA ACR pediatric 30 response at 48 weeks after the first daily administration. In some embodiments, the pediatric patient achieves a JIA ACR pediatric 50 response at 48 weeks after the first daily administration. In some embodiments, the pediatric patient achieves a JIA ACR pediatric 70 response at 48 weeks after the first daily administration. In some embodiments, the pediatric patient achieves a JIA ACR pediatric 90 response at 48 weeks after the first daily administration. In some embodiments, the pediatric patient achieves a JIA ACR pediatric 100 response at 48 weeks after the first daily administration.

In another aspect is provided a method for treating systemic juvenile idiopathic arthritis (SJIA) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In another aspect is provided a method for treating atopic dermatitis (AD) in a pediatric patient, the method comprising administering a therapeutically effective amount of upadacitinib to the pediatric. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient is less than twelve years old and has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient is less than twelve years old and has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient is less than twelve years old and has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient is less than twelve years old and has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient is less than twelve years old and has a body weight in a range from about 10 kg to less than about 20 kg, the method comprises administering upadacitinib twice daily at a dose of 3 mg each (3 mg BID); if the pediatric patient is less than twelve years old and has a body weight in a range from about 20 kg to less than about 30 kg, the method comprises administering upadacitinib twice daily at a dose of 4 mg each (4 mg BID); and if the pediatric patient is less than twelve years old and has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient is less than twelve years old and has a body weight in a range from about 10 kg to less than about 20 kg, the method comprises administering upadacitinib twice daily at a dose of 6 mg each (6 mg BID); if the pediatric patient is less than twelve years old and has a body weight in a range from about 20 kg to less than about 30 kg, the method comprises administering upadacitinib twice daily at a dose of 8 mg each (8 mg BID); and if the pediatric patient is less than twelve years old and has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient is less than twelve years old and has a body weight in a range from about 10 kg to less than about 20 kg, the method comprises administering upadacitinib twice daily at a dose of 6 mg each (6 mg BID); if the pediatric patient is less than twelve years old and has a body weight in a range from about 20 kg to less than about 30 kg, the method comprises administering upadacitinib twice daily at a dose of 8 mg each (8 mg BID); and if the pediatric patient is less than twelve years old and has a body weight of about 30 kg or greater, the method comprises administering upadacitinib twice daily at a dose of 12 mg each (12 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, if the pediatric patient is twelve years of age or older and has a body weight of less than about 40 kg, the method comprises administering upadacitinib twice daily at a dose of 8 mg each (8 mg BID) once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In some embodiments, the pediatric patient achieves an EASI 75 response at 12 weeks after the first daily administration. In some embodiments, the pediatric patient achieves an EASI 90 response at 12 weeks after the first daily administration. In some embodiments, the pediatric patient achieves an EASI 100 response at 12 weeks after the first daily administration. In some embodiments, the pediatric patient achieves an Investigator's Global Assessment scale for Atopic Dermatitis (vIGA-AD) score of 0 or 1 at 12 weeks after the first daily administration.

In another aspect is provided a method for treating juvenile psoriatic arthritis (JPsA) in a pediatric patient, the method comprising administering a therapeutically effective amount of upadacitinib to the pediatric. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dosc of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In another aspect is provided a method for treating juvenile ankylosing spondylitis (JAS) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In another aspect is provided a method for treating non-radiographic axial spondyloarthritis (nr-axSpA) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In another aspect is provided a method for treating hidradenitis suppurative (HS) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In another aspect is provided a method for treating systemic lupus erythematosus (SLE) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In a yet further aspect is provided a method for treating ulcerative colitis (UC) in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In a still further aspect is provided a method for treating Crohn's disease in a pediatric patient, the method comprising administering a therapeutically effective amount of upadacitinib to the pediatric patient. In some embodiments, the therapeutically effective amount of upadacitinib is administered to the pediatric patient as a stable liquid pharmaceutical composition. In some embodiments, the therapeutically effective amount of upadacitinib is administered to the pediatric patient as a solid dosage form.

In some embodiments, the pediatric patient has a body weight in a range from about 10 kg to less than about 20 kg, the method comprising administering a therapeutically effective amount of upadacitinib, wherein:
(i) the upadacitinib is administered twice daily at a dose of 3 mg each (3 mg BID); or
(ii) the upadacitinib is administered twice daily at a dose of 6 mg each (6 mg BID).

In some embodiments, the pediatric patient has a body weight in a range from about 20 kg to less than about 30 kg, the method comprising administering a therapeutically effective amount of upadacitinib to the pediatric patient, wherein:
(i) the upadacitinib is administered twice daily at a dose of 4 mg each (4 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 8 mg each (8 mg BID).

In some embodiments, the pediatric patient has a body weight of about 30 kg or greater, the method comprising administering a therapeutically effective amount of upadacitinib to the pediatric patient, wherein:
(i) the upadacitinib is administered twice daily at a dose of 6 mg each (6 mg BID); or
(ii) the upadacitinib is administered twice daily at a dose of 12 mg each (12 mg BID).

In some embodiments, if the pediatric patient has a body weight in a range from about 10 kg to less than about 20 kg, the method comprises administering upadacitinib twice daily at a dose of 3 mg each (3 mg BID); if the pediatric patient has a body weight in a range from about 20 kg to less than about 30 kg, the method comprises administering upadacitinib twice daily at a dose of 4 mg each (4 mg BID); and if the pediatric patient has a body weight of about 30 kg or greater, the method comprises administering upadacitinib twice daily at a dose of 6 mg each (6 mg BID) or once daily at a dose of 15 mg (15 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 8 mg each (8 mg BID) or once daily at a dose of 30 mg (30 mg QD).

In some embodiments, if the pediatric patient has a body weight in a range from about 10 kg to less than about 20 kg, the method comprises administering upadacitinib twice daily at a dose of 6 mg each (6 mg BID); if the pediatric patient has a body weight in a range from about 20 kg to less than about 30 kg, the method comprises administering upadacitinib twice daily at a dose of 8 mg each (8 mg BID); and if the pediatric patient has a body weight of about 30 kg or greater, the method comprises administering upadacitinib twice daily at a dose of 12 mg each (12 mg BID) or once daily at a dose of 30 mg (30 mg QD).

In some embodiments, the therapeutically effective amount of upadacitinib is administered to the pediatric patient as a stable oral pharmaceutical solution.

In some embodiments, the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

In some embodiments, the oral solution comprises upadacitinib, a buffer and/or pH adjusting agent, a preservative, a sweetener, and water.

In some embodiments, the oral solution comprises upadacitinib at a concentration of about 0.5 mg/mL.

In some embodiments, the oral solution comprises upadacitinib at a concentration of about 1 mg/mL.

In some embodiments, the pediatric patient has a body weight of about 30 kg or greater, the method comprising administering a therapeutically effective amount of upadacitinib to the pediatric patient, wherein:
(i) the upadacitinib is administered once daily at a dose of 15 mg (15 mg QD); or
(ii) the upadacitinib is administered once daily at a dose of 30 mg (30 mg QD).

In some embodiments, the therapeutically effective amount of upadacitinib is administered to the pediatric patient as an extended-release tablet.

In yet another aspect is provided a stable oral pharmaceutical formulation comprising upadacitinib or a pharmaceutically acceptable salt or solid-state form thereof, a buffer and/or pH adjusting agent, a preservative, a sweetener, and water.

In some embodiments, the stable oral pharmaceutical formulation comprises the anhydrous free base upadacitinib at a concentration of about 0.5 mg/mL.

In some embodiments, the stable oral pharmaceutical formulation comprises the anhydrous free base upadacitinib at a concentration of about 1 mg/mL.

In some embodiments, the buffer is selected from the group consisting of citrate, phosphate, tartrate, succinate, formate, acetate, and combinations thereof.

In some embodiments, the pH adjusting agent is selected from the group consisting of citric acid, phosphoric acid, tartaric acid, succinic acid, formic acid, acetic acid, and combinations thereof.

In some embodiments, the preservative is selected from the group consisting of sodium benzoate, benzoic acid, propyl paraben, sodium metabisulfite, potassium sorbate, para-hydroxybenzoic acid, para-hydroxybenzoate, and combinations thereof.

In some embodiments, the sweetener selected from the group consisting of sucralose, acesulfame potassium, sodium saccharin, neotame, sucrose, maltitol, xylitol, and combinations thereof.

In some embodiments, the stable oral pharmaceutical formulation comprises anhydrous free base upadacitinib, citric acid, sodium citrate, sodium benzoate, sucralose, and water.

In some embodiments, the stable oral pharmaceutical solution has a pH in a range from about 2 to about 5. In some embodiments, the stable oral pharmaceutical solution has a pH in a range from about 3 to about 4. In some embodiments, the stable oral pharmaceutical solution has a pH in a range from about 2.5 to about 3.5.

### BRIEF DESCRIPTION OF THE DRAWINGS

FIG. 1 is a schematic illustration of a clinical study in pediatric polyarticular course juvenile idiopathic arthritis patients according to a non-limiting embodiment of the disclosure.

FIG. 2 is a graphical depiction of mean upadacitinib plasma concentration-time profiles in pediatric polyarticular course juvenile idiopathic arthritis patients according to non-limiting embodiments of the disclosure.

FIGS. 3A-3E are graphical depictions of efficacy of upadacitinib at week 12 according to various measures and stratified by age group in pediatric polyarticular course juvenile idiopathic arthritis patients according to non-limiting embodiments of the disclosure.

FIG. 4 is a schematic illustration of a clinical study in pediatric atopic dermatitis patients according to a non-limiting embodiment of the disclosure.

FIGS. 5A to 5D are graphical depictions of mean upadacitinib plasma concentration-time profiles in pediatric atopic dermatitis patients according to non-limiting embodiments of the disclosure.

FIG. 6 is a schematic illustration of a clinical study in pediatric polyarticular course juvenile idiopathic arthritis patients according to a non-limiting embodiment of the disclosure.

FIG. 7 is a schematic illustration of patient disposition in the clinical study of Example

1.

FIGS. 8A to 8C are graphical depictions of mean upadacitinib plasma concentration-time profiles in pediatric polyarticular course juvenile idiopathic arthritis patients for various formulations according to non-limiting embodiments of the disclosure.

FIGS. 9A to 9E are a series of graphical depictions of efficacy of upadacitinib at week 12 according to various measures and stratified by age group in pediatric polyarticular course juvenile idiopathic arthritis patients according to non-limiting embodiments of the disclosure.

FIG. 10A is a graphical depiction of efficacy of upadacitinib over time through week 48 according to JIA ACR response rate in pediatric polyarticular course juvenile idiopathic arthritis patients according to non-limiting embodiments of the disclosure.

FIG. 10B is a graphical depiction of efficacy of upadacitinib over time through week 48 according to JADAS-27 [CRP] response rate in pediatric polyarticular course juvenile idiopathic arthritis patients according to non-limiting embodiments of the disclosure.

FIG. 10C is a graphical depiction of efficacy of upadacitinib over time through week 48 according to mean change in C-CHAQ from baseline in pediatric polyarticular course juvenile idiopathic arthritis patients according to non-limiting embodiments of the disclosure.

FIG. 10D is a graphical depiction of efficacy of upadacitinib over time through week 48 according to mean change in total number of active joints from baseline in pediatric polyarticular course juvenile idiopathic arthritis patients according to non-limiting embodiments of the disclosure.

### DETAILED DESCRIPTION OF THE DISCLOSURE

### I. Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

The term “AUC” refers to the area under the curve. AUC is the definite integral of a curve that describes the variation of a drug concentration in blood plasma as a function of time.

The term “Cₘₐₓ” refers to the plasma concentration of the referent drug at Tₘₐₓ, expressed herein as ng/ml, produced by the oral ingestion of a single dose, or indicated number of doses, of the dosage form or pharmaceutical composition, such as the dosage forms and compositions of the present disclosure. Unless specifically indicated, Cₘₐₓ refers to the overall maximum observed concentration.

The terms “treating”, “treatment”, and “therapy” and the like, as used herein, are meant to include therapeutic measures for a disease or disorder leading to any clinical desirable or beneficial effect, including but not limited to alleviation or relief of one or more symptoms, regression, slowing or cessation of progression of the disease or disorder.

As used herein, the term “pediatric patient” refers to a human patient of less than 18 years old. The terms “patient” and “subject” are used interchangeably herein.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art. Examples of pharmaceutically acceptable salts of upadacitinib may be found in WO 2017/066775, which is hereby incorporated by reference in its entirety.

### II. JAK1 Inhibition

Upadacitinib (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide), or a pharmaceutically acceptable salt or solid state form thereof, is an oral Janus kinase (JAK) inhibitor that displays unique selectivity for the JAK1 receptor. Upadacitinib has the structure shown below:

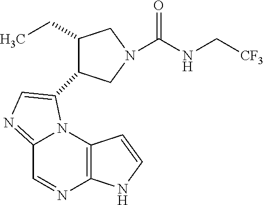

The dosage strength of upadacitinib recited in the present application is based on the weight of anhydrous freebase upadacitinib present in the active ingredient delivered to the patient. For example, a dose of “15 mg of upadacitinib” or “UPA 15 MG” refers to the 15 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “15 mg of upadacitinib” includes the administration of 15.4 mg of crystalline upadacitinib freebase hemihydrate (which includes ½ of a water conformer molecule per upadacitinib freebase molecule) which delivers 15 mg of anhydrous freebase upadacitinib to a patient. In another example, a dose of “30 mg of upadacitinib” or ““UPA 30 MG” refers to the 30 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “30 mg of upadacitinib” includes the administration of 30.7 mg of crystalline upadacitinib freebase hemihydrate (which includes ½ of a water conformer molecule per upadacitinib freebase molecule) which delivers 30 mg of anhydrous freebase upadacitinib to a patient.

### III. Pharmaceutical Compositions and Routes of Administration

Upadacitinib can be administered to a human patient by itself or in pharmaceutical composition where it is mixed with biologically suitable carriers or excipient(s) at doses to treat or ameliorate a disease or condition as described herein. Mixtures of these compounds can also be administered to the patient as a simple mixture or in suitable formulated pharmaceutical compositions.

The pharmaceutical compositions of the present disclosure may be manufactured in a manner that is itself known, e.g., by means of conventional mixing, dissolving, granulating, dragec-making, levigating, emulsifying, encapsulating, entrapping or lyophilizing processes.

Pharmaceutical compositions for use in accordance with the present disclosure thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

In some embodiments, the pharmaceutical composition is a capsule dosage form. In some embodiments, the pharmaceutical composition is a tablet dosage form. In some embodiments, tablet is a controlled-release formulation, such as an extended-release tablet dosage form (also referred to herein as a modified release or sustained release formulation Examples of solid dosage forms comprising upadacitinib may be found in WO 2017/066775, which is hereby incorporated by reference in its entirety.

In some embodiments, the composition is a stable liquid pharmaceutical composition. In some embodiments, the stable liquid pharmaceutical composition is a stable oral solution. In some embodiments, the stable liquid pharmaceutical composition is a stable oral suspension. Suitable stable liquid pharmaceutical compositions comprise upadacitinib or a pharmaceutically acceptable salt or solid-state form thereof, along with excipients such as buffers, preservatives, sweeteners, flavoring agents, pH adjusting agents, solvents, and the like. In some embodiments, the stable liquid pharmaceutical composition is a stable oral pharmaceutical solution comprising upadacitinib, a buffer and/or pH adjusting agent, a preservative, a sweetener, and water. In some embodiments, the stable liquid pharmaceutical composition is a stable oral pharmaceutical suspension comprising upadacitinib, a buffer and/or pH adjusting agent, a preservative, a sweetener, and water.

The concentration of upadacitinib in the stable liquid pharmaceutical composition may vary. Due to the bitterness of upadacitinib, which is difficult to mask at higher concentrations for palatability (see, e.g., Example 6), it is generally present at about 1 mg/mL or less. In some embodiments, the stable pharmaceutical composition is an oral solution comprising: upadacitinib at a concentration in a range from about 0.3 mg/mL to about 1.2 mg/mL, such as from about 0.3 to about 0.7 mg/mL, or from about 0.8 to about 1.2 mg/mL. In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL. In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises a pH adjusting agent. Suitable pH adjusting agents include acids such as mineral or organic acids. Mineral acids include but are not limited to hydrochloric, sulfuric, and phosphoric acid. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H). In some embodiments, the pH adjusting agent is an organic acid. Suitable organic acids include, but are not limited to, benzoic acid, toluic acids, salicylic acid, benzenesulfonic acid, p-toluenesulfonic acid, 2-(4-isobutylphenyl)propanoic acid, 2,2-dichloroacetic acid, 2-hydroxyethancsulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, citric acid, cyclamic acid, dodecylsulfuric acid, ethane-1,2-disulfonic acid, ethanesulfonic acid, fumaric acid, furoic acid, galactaric acid, gentisic acid, glucoheptonic acid, gluconic acid, glucuronic acid, glutamic acid, glutaric acid, glycerophosphoric acid, glycolic acid, hippuric acid, isobutyric acid, isovaleric acid, lactobionic acid, lauric acid, levulinic acid, malic acid, maleic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, tartaric acid, and undecylenic acid. In some embodiments, the pH adjusting agent is selected from the group consisting of citric acid, phosphoric acid, tartaric acid, succinic acid, formic acid, acetic acid, and combinations thereof. In some embodiments, the pH adjusting agent is citric acid.

In some embodiments, the stable liquid pharmaceutical composition comprises a buffer. Suitable buffers include, but are not limited to, citrate, phosphate, tartrate, succinate, glycinate, glycerophosphate, formate, and acetate. In some embodiments, the buffer is selected from the group consisting of citrate, phosphate, tartrate, succinate, formate, acetate, and combinations thereof. In some embodiments, the buffer is selected from the group consisting of citrate and phosphate. In some embodiments, the buffer is sodium citrate.

The quantity of pH adjusting agent and/or buffer may vary. Generally, the concentration of each is adjusted to provide a desired pH range of the resulting stable liquid pharmaceutical composition. In some embodiments, the stable liquid pharmaceutical composition has a pH in a range from about 2 to about 5, or from about 3 to about 4, or from about 2 to about 3, or from about 4 to about 5, or from about 2.0 to about 2.5, or from about 2.5 to about 3.0, or from about 3.0 to about 3.5, or from about 3.5 to about 4.0, or from about 4.0 to about 4.5, or from about 4.5 to about 5.0, or from about 3.0 to about 3.1, or from about 3.0 to about 3.2, or from about 3.0 to about 3.3, or from about 3.1 to about 3.2, or from about 3.1 to about 3.3, or from about 3.1 to about 3.4, or from about 3.1 to about 3.5, or from about 3.2 to about 3.3, or from about 3.2 to about 3.4, or from about 3.2 to about 3.5, or from about 3.3 to about 3.4, or from about 3.3 to about 3.5. In some embodiments, the stable liquid pharmaceutical composition has a pH of about 3.0, or about 3.1, or about 3.2.

In some embodiments, the stable liquid pharmaceutical composition comprises a preservative. Suitable preservatives include, but are not limited to, benzoic acid, sodium benzoate, benzyl alcohol, ascorbic acid, potassium sorbate, 4-hydroxybenzoic acid, 4-hydroxybenzoate, methyl paraben, propyl paraben, sodium metabisulfite, and combinations thereof. In some embodiments, the preservative is selected from the group consisting of sodium benzoate, benzoic acid, propyl paraben, sodium metabisulfite, potassium sorbate, para-hydroxybenzoic acid, para-hydroxybenzoate, and combinations thereof.

In some embodiments, the stable liquid pharmaceutical composition comprises a sweetener. The sweetener can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is selected from the group consisting of sucralose, acesulfame potassium, sodium saccharin, neotame, sucrose, maltitol, xylitol, and combinations thereof.

In some embodiments, the stable oral pharmaceutical solution comprises one or more flavoring agents. Any flavorful or aromatic substance capable of altering the taste, fragrance, or both of the solution may be utilized. Flavoring agents may be natural or synthetic. Suitable flavoring agents include, but are not limited to, flavor packages imparting flavors such as cherry, orange, lemon, lime, bubblegum, grape, strawberry, mango, and the like.

In some embodiments, the stable oral pharmaceutical solution comprises a taste modifier. Suitable taste modifiers include, but are not limited to, salts such as sodium chloride, and monoammonium glycyrrhizinate to accentuate sweetness.

In some embodiments, the stable pharmaceutical composition is an oral solution comprising upadacitinib, citric acid, sodium citrate, sodium benzoate, sweetener, and water.

In some embodiments, the stable pharmaceutical composition comprises citric acid in an amount in a range from about 0.1 to about 1 mg/mL.

In some embodiments, the stable pharmaceutical composition comprises sodium citrate in an amount in a range from about 0.01 to about 1 mg/mL.

In some embodiments, the stable pharmaceutical composition comprises sodium benzoate in an amount in a range from about 0.01 to about 0.1 mg/mL.

In some embodiments, the stable pharmaceutical composition comprises a sweetener in an amount in a range from about 1 to about 50 mg/mL.

In particular embodiments, the stable pharmaceutical composition is an oral solution having the formulation provided in Table 1.

TABLE 1
Formulation of upadacitinib oral solution (1 mg/mL)

| Component | mg/mL |
|---|---|
| upadacitinib | 0.3-1.2 |
| citric acid, anhydrous | 0.15-0.35 |
| sodium citrate, dihydrate | 0.03-0.06 |
| sodium benzoate | 0.02-0.06 |
| sucralose | 7-11 |
| purified water | q.s to 1 mL |

The pH of the stable oral solution may vary. In some embodiments, the stable oral solution has a pH in a range from about 2 to about 5. In some embodiments, the stable oral solution has a pH in a range from about 3 to about 4. In some embodiments, the stable oral solution has a pH in a range from about 2.5 to about 3.5.

### IV. Pediatric Dosing

In some embodiments, the pediatric patient has an age of less than 18 years. In some embodiments, the pediatric patient has an age of less than 12 years. In some embodiments, the pediatric patient has an age of less than 6 years. In some embodiments, the pediatric patient has an age in a range from about 2 to less than about 6 years, in a range from about 6 to less than about 12 years, or in a range from about 12 to less than about 18 years. In some embodiments, the pediatric patient has an age in a range from about 2 to about 18 years, such as about 2, about 3, about 4, about 5, about 6, about 7, about 8, about 9, about 10, about 11, about 12, about 13, about 14, about 15, about 16, about 17, or about 18 years of age. In some embodiments, the pediatric patient is two years of age or older. In some embodiments, the pediatric patient has an age in a range from about 2 to less than 12 years old.

In some embodiments, the pediatric patient has a body weight of at least about 10 kg. In some embodiments, the pediatric patient has a body weight from about 10 kg to less than about 30 kg, such as from about 20 to less than about 20 kg, or from about 20 to less than about 30 kg. In some embodiments, the pediatric patient has a body weight 30 kg or more. In some embodiments, the pediatric patient has an age in a range from about 2 to less than 12 years old and weighs less than 40 kg. In some embodiments, the pediatric patient has an age of 12 years old or greater and weighs less than 40 kg.

In some embodiments, the pediatric patient has a body weight in a range from about 10 to less than about 20 kg, the method comprising administering 3 mg of upadacitinib twice daily (3 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 3 mg dose is provided BID as about 3 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 3 mg dose is provided BID as about 6 mL of the about 0.5 mg/mL

### Solution

In some embodiments, the pediatric patient has a body weight in a range from about 10 to less than about 20 kg, the method comprising administering 6 mg of upadacitinib twice daily (6 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 6 mg dose is provided BID as about 6 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the pediatric patient has a body weight in a range from about 20 to less than about 30 kg, the method comprising administering 8 mg of upadacitinib twice daily (8 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution.

In some embodiments, the pediatric patient has a body weight of about 30 kg or greater, the method comprising administering 12 mg of upadacitinib twice daily (12 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 6 mg dose is provided BID as about 6 mL of the about 1 mg/mL solution.

In some embodiments, the oral solution comprises upadacitinib at a concentration of about 1 mg/mL, and the 12 mg dose is provided BID as about 12 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 6 mg dose is provided BID as about 12 mL of the about 0.5 mg/ml solution.

In some embodiments, the oral solution comprises upadacitinib at a concentration of about 0.5 mg/mL and the 12 mg dose is provided BID as about 24 mL of the about 0.5 mg/ml solution.

In some embodiments, the pediatric patient has a body weight of about 30 kg or greater, the method comprising administering 15 mg of upadacitinib once daily (15 mg QD) as an extended-release tablet.

In some embodiments, the pediatric patient has a body weight of about 30 kg or greater, the method comprising administering 8 mg of upadacitinib twice daily (8 mg BID) as an oral solution.

In some embodiments, the pediatric patient has a body weight of about 30 kg or greater, the method comprising administering 12 mg of upadacitinib twice daily (12 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 30 mg of upadacitinib once daily (30 mg QD) as an extended-release tablet.

The maximal concentration achieved (Cₘₐₓ) with the aforementioned dosing may vary depending on e.g., the dose, the weight of the patient, and the individual patient's metabolism of upadacitinib.

In some embodiments, when an immediate release oral solution as described herein is administered BID to the pediatric subject, a mean Cₘₐₓ for upadacitinib is achieved in a range from about 20 to about 160 ng/mL.

In some embodiments, when administered twice daily at a dose of 3 or 4 mg each (3 or 4 mg BID) to the pediatric subject, a mean Cₘₐₓ for upadacitinib is achieved in a range from about 25 to about 50 ng/ml, such as from about 25 to about 35, from about 25 to about 33, from about 25 to about 31, from about 25 to about 29, or from about 25 to about 27 ng/mL.

In some embodiments, when administered twice daily at a dose of 6 or 8 mg each (6 or 8 mg BID) to the pediatric subject, a mean Cₘₐₓ for upadacitinib is achieved in a range from about 40 to about 100 ng/ml, such as from about 40 to about 95, from about 40 to about 90, from about 40 to about 85, from about 40 to about 80, from about 40 to about 75, from about 40 to about 70, from about 40 to about 65, from about 40 to about 60, from about 40 to about 55, from about 40 to about 50, or from about 40 to about 45 ng/mL.

In some embodiments, when an extended-release 15 mg tablet as described herein is administered once daily (15 mg QD) to the pediatric subject, a mean Cₘₐₓ for upadacitinib is achieved in a range from about 45 to about 50 ng/ml, such as from about 45 to about 49, from about 45 to about 48, from about 45 to about 46, or from about 45 to about 46 ng/mL.

In some embodiments, when an extended-release 30 mg tablet as described herein is administered once daily (30 mg QD) to the pediatric subject, a mean Cₘₐₓ for upadacitinib is achieved in a range from about 150 to about 160 ng/ml, such as from about 152 to about 159, from about 153 to about 158, from about 154 to about 156, or from about 154 to about 155 ng/mL.

The mean 24-hour exposure achieved (AUC₀₋₂₄) with the aforementioned dosing may vary, depending on e.g., the dose, the weight of the patient, the fed vs. fasted condition, and the individual patient's metabolism of upadacitinib.

In some embodiments, when an immediate release oral solution as described herein is administered BID to the pediatric subject, a mean AUC₀₋₂₄ for upadacitinib is achieved in a range from about 200 to about 700 ng·h/mL.

In some embodiments, when administered twice daily at a dose of 3 or 4 mg each (3 or 4 mg BID) to the pediatric subject, a mean AUC₀₋₂₄ for upadacitinib is achieved in a range from about, such as from about 220 to about 270 ng·h/mL, such as from about 220 to about 265, from about 220 to about 260, from about 220 to about 255, from about 220 to about 250, from about 220 to about 245, from about 220 to about 240, from about 220 to about 235, from about 220 to about 230, or from about 220 to about 225 ng·h/mL.

In some embodiments, when administered twice daily at a dose of 6 or 8 mg each (6 or 8 mg BID) to the pediatric subject, a mean AUC₀₋₂₄ for upadacitinib is achieved in a range from about 340 to about 590 ng·h/mL, such as from about 340 to about 580, from about 340 to about 570, from about 340 to about 560, from about 340 to about 550, from about 340 to about 540, from about 340 to about 530, from about 340 to about 520, from about 340 to about 510, from about 340 to about 500, from about 340 to about 490, from about 340 to about 480, from about 340 to about 470, from about 340 to about 460, from about 340 to about 450, from about 340 to about 440, from about 340 to about 430, from about 340 to about 420, from about 340 to about 410, from about 340 to about 400, from about 340 to about 390, from about 340 to about 380, from about 340 to about 370, from about 340 to about 360, from about 340 to about 350, or from about 340 to about 345 ng·h/mL. In some embodiments, when administered twice daily at a dose of 6 or 8 mg each (6 or 8 mg BID) to the pediatric subject, a mean AUC₀₋₂₄ for upadacitinib is achieved in a range from about 570 to about 590 ng·h/mL, such as from about 570 to about 590, from about 570 to about 588, from about 570 to about 586, from about 570 to about 584, from about 570 to about 582, or from about 570 to about 580 ng·h/mL.

In some embodiments, when an extended-release 15 mg tablet as described herein is administered once daily (15 mg QD) to the pediatric subject, a mean AUC₀₋₂₄ for upadacitinib is achieved in a range from about 45 to about 50 ng·h/mL, such as from about 45 to about 49, about 46 to about 48, or from about 47 to about 48 ng·h/mL.

In some embodiments, when an extended-release 30 mg tablet as described herein is administered once daily (30 mg QD) to the pediatric subject, a mean AUC₀₋₂₄ for upadacitinib is achieved in a range from about 650 to about 680 ng/ml, such as from about 660 to about 670, or from about 665 to about 670 ng·h/mL.

The disclosed methods generally comprise orally administering the upadacitinib to the patient daily for a period of time. In some embodiments, the administration is continued at the same dose and dosing frequency over a treatment period. The duration of the treatment period may vary. For example, the treatment period may be at least 14 days, at least one month, 3 months, 4 months, 6 months, 9 months, 1 year, 2 years, 5 years, 10 years, 20 years, 50 years, or more. In some embodiments, the treatment period is 12 weeks. In some embodiments, the treatment period is 156 weeks. In some embodiments, the treatment period is at least 156 weeks.

### V. Treatment of Pediatric Patients with Polyarticular Course Juvenile Idiopathic Arthritis

In some embodiments, the pediatric patient has polyarticular course juvenile idiopathic arthritis (pcJIA), including rheumatoid factor-positive or rheumatoid factor-negative polyarticular JIA, extended oligoarticular JIA, or systemic JIA with active arthritis and without active systemic features.

Non-steroidal anti-inflammatory drugs (NSAIDs) are the mainstay of treatment in JIA, since they are believed to be the least toxic agent in children. They provide symptomatic relief but are not considered to be disease modifying. Disease-modifying anti-rheumatic drugs (DMARDs) such as methotrexate (MTX) and sulfasalazine are effective in JIA whereas hydroxychloroquine, D-penicillamine, and auranofin are not. Most children respond to MTX therapy, which has acceptable toxicity although remission is rare. To a variable extent, systemic use of corticosteroids is frequent in all JIA conditions but particularly for JIA is less desirable due to many deleterious effects. Systemic and intra-articular corticosteroids are being used in JIA in conjunction with NSAIDs and DMARDs. Intra-articular (IA) corticosteroid injections are recommended in JIA patients who have active arthritis regardless of the use of additional concomitant therapy. IA steroids, however, are frequently used and often induce prolonged remission in children with oligoarticular JIA.

Many potent biologic agents are now available for use in the treatment of JIA and are capable of inducing remission in JIA when used as monotherapy or in combination with MTX or other synthetic DMARDs. However, many patients still do not reach a state of remission or low disease activity with these agents or lose response over time. Additionally, given the potential safety concerns associated with the immunomodulatory effects of biologic agents, and given the fact that all biologic agents are administered by injection, novel oral treatment options with an improved benefit/risk profile are warranted to treat JIA. Accordingly, it would be desirable to provide pediatric patients with an alternate therapy for treatment of pcJIA.

JAK inhibition is known to inhibit the IL-6 pathway, and IL-6 in turn is known to be involved in the pathogenesis of both RA and juvenile idiopathic arthritis (JIA). See, e.g., Ou et al. Clin Rheumatol. 2002, 21:52-6; Mangge et al. Arthritis Rheum. 1995, 38(2):211-20; and Mellins ct al. Nat Rev Rheumatol. 2011, 7(7):416-26. In particular, inhibition of the JAK1 subtype blocks the signaling of many important pro-inflammatory cytokines, including interleukin (IL)-2, IL-6, IL-7, and IL-15, which are known contributors to inflammatory disorders. Through modulation of these pro-inflammatory cytokine pathways, upadacitinib offers the potential for effective treatment of inflammatory or autoimmune disorders. Without wishing to be bound by any particular theory, it is believed that, based on the differentiated selectivity profile for JAK inhibition, upadacitinib could demonstrate an improved benefit/risk profile compared to other less selective JAK inhibitors or other therapeutic strategies for patients with inflammatory diseases. In particular, it is believed that upadacitinib will provide therapeutic benefit in pcJIA.

In adults, model-estimated area under the plasma upadacitinib concentration curve (AUC) over 0-24 hours (AUC₀₋₂₄) at steady state were 362 and 720 ng·h/mL after multiple 15 mg and 30 mg once daily (QD) doses, respectively. Accordingly, pediatric doses achieving such exposures are disclosed herein as described above.

As disclosed herein, a prototype oral solution formulation was developed to allow suitable and flexible dosing in younger pediatric patients. Administration of two 6 mg doses of upadacitinib oral solution (1 mg/mL) 12 hours apart resulted in approximately 30% and 20% higher Cₘₐₓ and AUC, respectively, relative to a single 15 mg QD upadacitinib (used in Phase 3 RA studies) under fasting conditions; these results supported the selection of upadacitinib doses in pediatric subjects in the study of Example 1.

Accordingly, in one aspect is provided a method of treating pcJIA in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL

### Solution

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/ml solution.

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a history of arthritis affecting at least 5 joints within the first 6 months of disease (for extended oligoarticular JIA: ≤4 joints during the first 6 months of disease and >4 joints thereafter), as per International League of Associations for Rheumatology (ILAR) criteria.

In some embodiments, the pediatric patient does not have a diagnosis of enthesitis-related arthritis (ERA) or juvenile psoriatic arthritis (JPSA).

In some embodiments, the pediatric patient has 5 or more active joints, defined as the presence of swollen joints (not due to deformity) or, in the absence of swelling, joints with limitation of movement (LOM) plus pain on motion and/or tenderness with palpation, with LOM present in at least three of the active joints.

In some embodiments, the pediatric patient is receiving methotrexate. In such embodiments, the patient should be on a stable dose of ≤20 mg/m2 for at least 8 weeks before the start of administration.

In some embodiments, the pediatric patient is receiving oral glucocorticoids. In such embodiments, the patient should be on a stable dose (no greater than 10 mg/day or 0.2 mg/kg/day, whatever is lower) for at least 1 week before the start of administration.

In some embodiments, the patient achieves one or more of a JIA ACR pediatric 30/50/70/90/100 response, a change from baseline in JADAS10/27/71 responses, low disease activity or remission according to JADAS-based criteria. In some embodiments, the patient achieves one or more of a JIA ACR pediatric 30/50/70/90/100 response at 12 weeks, 24 weeks, or 48 weeks.

In some embodiments, the pediatric patient does not have any of ongoing or active uveitis within 3 months prior to initiating treatment, active TB active infection(s) requiring treatment with parenteral anti-infectives, chronic recurring infection and/or active viral infection, active hepatitis B virus (HBV) or hepatitis C virus (HCV) infection, a positive result of beta-D-glucan.

In some embodiments, the pediatric patient has not been treated with intra-articular or parenteral administration of corticosteroids in the preceding 4 weeks prior to the start of administration.

In some embodiments, the pediatric patient has no history of: any malignancy except for successfully treated non-melanoma skin cancer or localized carcinoma in situ of the cervix, recurrent or disseminated (even a single episode) herpes zoster; disseminated (even a single episode) herpes simplex; human immunodeficiency virus (HIV) infection; previous reception of an organ transplant that requires continued immunosuppression, gastrointestinal perforation (other than appendicitis or penetrating injury), diverticulitis, or significantly increased risk for gastrointestinal perforation; prior exposure to a JAK inhibitor.

In some embodiments, the pediatric patient is not using known moderate or strong inhibitors (e.g., amiodarone, clarithromycin, fluconazole, ciprofloxacin, itraconazole, ketoconazole, quinidine, fluoxetine, and paroxetine) or inducers (e.g., carbamazepine, rifampin, phenobarbital, and phenytoin) of drug metabolizing enzymes.

In some embodiments, the pediatric patient is not using biologic treatment (etanercept, infliximab, adalimumab, abatacept, golimumab, tocilizumab, ustekinumab, certolizumab pegol, canakinumab, anakinra).

In some embodiments, the pediatric patient is not using a JAK inhibitor (e.g., commercially available upadacitinib [Rinvoq®], tofacitinib [Xeljanz®], ruxolitinib [Jakafi®], baricitinib [Olumiant®], peficitinib [Smyraf®], abrocitinib [PF-04965842], or filgotinib).

In some embodiments, the pediatric patient has moderately to severely active pcJIA.

In some embodiments, the pediatric patient has had an inadequate response to one or more DMARDs.

In some embodiments, the pediatric patient has had an inadequate response to one or more TNF blockers.

### VI. Treatment of Pediatric Patients with Systemic Juvenile Idiopathic Arthritis

In some embodiments, the pediatric patient has systemic juvenile idiopathic arthritis (SJIA), which may also be referred to as Still's disease or systemic juvenile rheumatoid arthritis. SJIA is officially a subset of juvenile idiopathic arthritis (JIA), although the pathophysiology is most consistent with an autoinflammatory disorder. SJIA affects not only the joints but other parts of the body, including the liver, lungs and heart. The course of SJIA is highly variable, typically initially manifesting with a several-month period of spiking fevers and rash, with varying degrees of arthralgia and arthritis. Currently, there is no cure for SJIA but remission is possible. Traditionally, a typical treatment goal for children with SJIA included relieving pain and controlling symptoms utilizing nonsteroidal anti-inflammatory drugs (NSAIDs) and/or high doses of oral or IV corticosteroids. More recently, biologic and nonbiologic discasc-modifying antirheumatic drugs (DMARDs) have become available. However, it remains desirable in the art to provide pediatric patients with alternate safe, well tolerated, and efficacious nonbiologic DMARDS therapies for treatment of SJIA.

JAK inhibition is known to inhibit the IL-6 pathway, and IL-6 in turn is known to be involved in the pathogenesis of both RA and juvenile idiopathic arthritis (JIA). See, e.g., Ou et al. Clin Rheumatol. 2002, 21:52-6; Mangge et al. Arthritis Rheum. 1995, 38(2):211-20; and Mellins ct al. Nat Rev Rheumatol. 2011, 7(7):416-26. In particular, inhibition of the JAK1 subtype blocks the signaling of many important pro-inflammatory cytokines, including interleukin (IL)-2, IL-6, IL-7, and IL-15, which are known contributors to inflammatory disorders. Through modulation of these pro-inflammatory cytokine pathways, upadacitinib offers the potential for effective treatment of inflammatory or autoimmune disorders. Without wishing to be bound by any particular theory, it is believed that, based on the differentiated selectivity profile for JAK inhibition, upadacitinib could demonstrate an improved benefit/risk profile compared to other less selective JAK inhibitors or other therapeutic strategies for patients with inflammatory diseases. In particular, it is believed that upadacitinib will provide therapeutic benefit in SJIA.

Pediatric doses achieving exposures consistent with efficacy in adult patients are disclosed herein as described above (i.e., based on body weight). Accordingly, in one aspect is provided a method of treating SJIA in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL

### Solution

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/ml solution.

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has moderately to severely active sJIA.

In some embodiments, the pediatric patient has had an inadequate response to one or more DMARDs.

In some embodiments, the pediatric patient has had an inadequate response to one or more TNF blockers.

### VII. Treatment of Pediatric Patients with Atopic Dermatitis

In some embodiments, the pediatric patient has Atopic Dermatitis (AD; also known as atopic eczema). AD is an inflammatory, pruritic, chronic or chronically relapsing skin disease. Common clinical characteristics include erythema, edema, xerosis, crosions/excoriations, oozing and crusting, and lichenification, but they vary by patient age and chronicity of lesions. Pruritus is a hallmark of the condition that is responsible for much of the disease burden borne by patients and their families (Williams, N. Engl. J. Med. 2005, 352(22):2314-24). AD is one of the most common skin diseases which affects up to 20% of children. In approximately 70% of cases, the onset of AD starts in children under 5 years of age (Williams et al., Atopic dermatitis: the epidemiology, causes, and prevention of atopic eczema. Cambridge, United Kingdom: Cambridge University Press, 2000:41-59). Long-term oral treatment options are limited for patients with moderate to severe AD recalcitrant to topical therapy; most conventional oral immunosuppressive therapies are used off-label, have limited efficacy data, and are not suitable for long-term treatment owing to their safety profiles. Accordingly, it is desirable in the art to provide pediatric patients with safe, well tolerated, and efficacious therapies for treatment of AD.

Pediatric doses (i.e., based on body weight) achieving exposures consistent with efficacy in adult patients are disclosed herein as described above and in Example 1 below. Accordingly, in one aspect is provided a method of treating AD in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of AD in an adult patient.

In some embodiments, the pediatric patient is less than twelve years old and has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/mL solution.

In some embodiments, the pediatric patient is less than twelve years old and has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient is less than twelve years old and has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/mL solution.

In some embodiments, the pediatric patient is less than twelve years old and has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient is less than twelve years old and has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient is less than twelve years old and has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient is less than twelve years old and has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient is less than twelve years old and has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient is twelve years of age or older and has a body weight of less than about 40 kg, the method comprises administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient is twelve years of age or older and has a body weight of less than about 40 kg, the method comprises administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient achieves one or more of a validated Investigator's Global Assessment scale for Atopic Dermatitis (vIGA-AD) score of 0 or 1, or an Eczema Arca and Severity Index by at least 75% (EASI 75), 90% (EASI 90) or 100% (EASI 100). In some embodiments, the pediatric patient achieves one or more of a validated Investigator's Global Assessment scale for Atopic Dermatitis (vIGA-AD) score of 0 or 1, or an Eczema Arca and Severity Index by at least 75% (EASI 75), 90% (EASI 90) or 100% (EASI 100) is achieved at 8 weeks, 10 weeks, 12 weeks, 14 weeks, 16 weeks, 18 weeks, 20 weeks, 22 weeks, 24 weeks, 48 weeks, or 52 weeks after the first daily administration. In some embodiments, the pediatric patient achieves an Eczema Area and Severity Index 75 at 12 weeks after the first daily administration. In some embodiments, the pediatric patient achieves an Eczema Arca and Severity Index 90 at 12 weeks after the first daily administration. In some embodiments, the pediatric patient achieves an Eczema Arca and Severity Index 100 at 12 weeks after the first daily administration. In some embodiments, the pediatric patient achieves an Investigator's Global Assessment scale for Atopic Dermatitis (vIGA-AD) score of 0 or 1 at 12 weeks after the first daily administration. In some embodiments, the pediatric patient achieves an Investigator's Global Assessment scale for Atopic Dermatitis (vIGA-AD) score of 0 at 12 weeks after the first daily administration. In some embodiments, the pediatric patient achieves an Investigator's Global Assessment scale for Atopic Dermatitis (vIGA-AD) score of 1 at 12 weeks after the first daily administration.

In some embodiments, the pediatric patient has severe AD.

In some embodiments, the pediatric patient has moderately to severely active pcJIA.

In some embodiments, the pediatric patient has had an inadequate response to one or more TCS.

In some embodiments, the pediatric patient has had an inadequate response to one or more biologic therapy.

### VIII. Treatment of Pediatric Patients with Juvenile Psoriatic Arthritis

In some embodiments, the pediatric patient has juvenile psoriatic arthritis (JPsA). JPsA is a chronic systemic inflammatory disease classified as a sub-type of spondyloarthritis (SpA) and characterized by the association of arthritis and psoriasis. The course of JPsA is usually characterized by flares and remissions. Left untreated, patients with JPsA can have persistent inflammation, progressive joint damage, disability, and a reduced life expectancy. Initial treatment of the musculoskeletal symptoms is composed of nonsteroidal anti-inflammatory drugs (NSAIDs) and local corticosteroid injections, while topical therapies are used for the initial treatment of psoriasis. For subjects who experience lack of efficacy or toxicity with these measures, systemic therapy with non-biologic disease modifying anti-rheumatic drugs (non-biologic DMARDs) (e.g., methotrexate [MTX], leflunomide [LEF], sulfasalazine [SSZ]) and ciclosporin A, followed by anti-tumor necrosis factor (TNF) therapy in subjects who do not respond adequately, is recommended. Other biologic therapies (e.g., IL-12/23 or IL-17 inhibitors) are also recommended as alternatives to anti-TNF inhibitors in selected PsA subjects. See, e.g., Gossec et al., Ann Rheum Dis. (2016) 75:499-510; Coates et al., Arthritis Rheumatol. (2016) 68:1060-71. However, despite the beneficial results achieved with currently available biologic agents, approximately 40% of patients do not have at least 20% improvement in American College of Rheumatology (ACR) scores and only 58% to 61% of patients with PsA who receive them are able to achieve clinical remission after 1 year of treatment, with only approximately 43% achieving sustained remission for at least 1 year. See, e.g., Gossec et al., Ann Rheum Dis. (2016) 75:499-510; Alamanos et al., J Rheumatol. (2003) 30:2641-2644; Savolainen et al., J Rheumatol. (2003) 30:2460-8; Sandborn, Dig Dis. (2010) 28:536-42; Saber et al., Arthritis Res Therapy (2010) 12: R94; Perrotta et al., J Rheumatol. (2016) 43:350-5.

Thus, there continues to remain a clear medical need for additional therapeutic options for the treatment of juvenile psoriatic arthritis (JPsA). Accordingly, it is desirable in the art to provide pediatric patients with safe, well tolerated, and efficacious therapies for treatment of JPsA.

Pediatric doses (i.e., based on body weight) achieving exposures consistent with efficacy in adult patients are disclosed herein as described above and in Example 1 below. Accordingly, in one aspect is provided a method of treating JPsA in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of JPsA in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/mL

### Solution

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL

### Solution

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/mL

### Solution

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has moderately to severely active JPsA.

In some embodiments, the pediatric patient has had an inadequate response to one or more DMARDs.

In some embodiments, the pediatric patient has had an inadequate response to one or more TNF blockers.

### IX. Treatment of Pediatric Patients with Juvenile Ankylosing Spondylitis

In some embodiments, the pediatric patient has juvenile ankylosing spondylitis (JAS). JAS is a chronic, inflammatory rheumatic disease primarily affecting the axial skeleton, characterized by chronic back pain (including nocturnal back pain), morning stiffness, enthesitis, peripheral arthritis, and extra-articular manifestations.

Due to the longstanding debilitating nature of AS, irreversible structural damage often occurs, negatively impacting patients' lives. No cure for AS exists, thus the primary goal of treatment is to maximize patients' quality of life through controlling the signs and symptoms of disease, preventing structural damage, and maintaining physical function, ideally by achieving sustained clinical remission or, at minimum, low disease activity. Nonsteroidal anti-inflammatory drugs (NSAIDs) are the first-line treatment for AS, followed by biologic disease-modifying antirheumatic drugs (bDMARDs), such as tumor necrosis factor (TNF) inhibitors or interleukin-17 (IL-17) inhibitors, in patients who do not sufficiently respond to NSAIDs. TNF inhibitors and IL-17 inhibitors are efficacious in some patients with AS, but there are still patients for whom neither of these approved therapies address individual treatment goals. AS is a difficult disease to treat, as shown based on low efficacy achieved with IL-6 inhibitors tocilizumab and sarilumab, as well as IL-12/23 inhibitor ustekinumab and T cell blockade inhibitor abatacept. See, e.g., Sieper et al., Ann. Rheum. Dis. 2014, 73:95-100, Sieper et al., Ann. Rheum. Dis. 2015, 74:1051-1057; Deodhar et al., Arthritis and Rheumatology 2019, 71:258-270, and Song et al., Ann. Rheum. Dis. 2011, 70:1108-1110.

Thus, there continues to remain a clear medical need for additional therapeutic options for the treatment of juvenile ankylosing spondylitis (JAS). Accordingly, it is desirable in the art to provide pediatric patients with safe, well tolerated, and efficacious therapies for treatment of AS.

Pediatric doses (i.e., based on body weight) achieving exposures consistent with efficacy in adult patients are disclosed herein as described above and in Example 1 below. Accordingly, in one aspect is provided a method of treating JAS in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of JAS in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/mL

### Solution

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/ml solution.

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL

### Solution

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has moderately to severely active JAS.

In some embodiments, the pediatric patient has had an inadequate response to one or more DMARDs.

In some embodiments, the pediatric patient has had an inadequate response to one or more TNF blockers.

### X. Treatment of Pediatric Patients with Non-Radiographic Axial Spondyloarthritis

In some embodiments, the pediatric patient has non-radiographic axial spondyloarthritis (nr-axSpA). Nr-axSpA is a chronic, inflammatory rheumatic disease primarily affecting the axial skeleton, characterized by chronic back pain (including nocturnal back pain), morning stiffness, enthesitis, peripheral arthritis, and extra-articular manifestations, nr-axSpA is the “early” form of ankylosing spondylitis (AS) and shares many of the same characteristics.

Due to the longstanding debilitating nature of nr-axSpA, irreversible structural damage often occurs, negatively impacting patients' lives. No cure for nr-axSpA exists, thus the primary goal of treatment is to maximize patients' quality of life through controlling the signs and symptoms of disease, preventing structural damage, and maintaining physical function, ideally by achieving sustained clinical remission or, at minimum, low disease activity. Nonsteroidal anti-inflammatory drugs (NSAIDs) are the first-line treatment for AS, followed by biologic disease-modifying antirheumatic drugs (bDMARDs), such as tumor necrosis factor (TNF) inhibitors or interleukin-17 (IL-17) inhibitors, in patients who do not sufficiently respond to NSAIDs. TNF inhibitors and IL-17 inhibitors are efficacious in some patients with AS, but there are still patients for whom neither of these approved therapies address individual treatment goals. AS is a difficult disease to treat, as shown based on low efficacy achieved with IL-6 inhibitors tocilizumab and sarilumab, as well as IL-12/23 inhibitor ustekinumab and T cell blockade inhibitor abatacept. Sec, e.g., Sieper et al., Ann. Rheum. Dis. 2014, 73:95-100, Sieper et al., Ann. Rheum. Dis. 2015, 74:1051-1057; Deodhar et al., Arthritis and Rheumatology 2019, 71:258-270, and Song et al., Ann. Rheum. Dis. 2011, 70:1108-1110. Accordingly, it is desirable in the art to provide pediatric patients with safe, well tolerated, and efficacious therapies for treatment of nr-axSpA.

Pediatric doses (i.e., based on body weight) achieving exposures consistent with efficacy in adult patients are disclosed herein as described above and in Example 1 below. Accordingly, in one aspect is provided a method of treating nr-axSpA in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of nr-axSpA in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/ml solution.

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has moderately to severely active nr-axSpA.

In some embodiments, the pediatric patient has had an inadequate response to one or more DMARDs.

In some embodiments, the pediatric patient has had an inadequate response to one or more TNF blockers.

### XI. Treatment of Pediatric Patients with Hidradenitis Suppurativa

In some embodiments, the pediatric patient has hidradenitis suppurative (HS). HS is a debilitating skin disorder of the apocrine glands (sweat glands found on certain parts of the body) and hair follicles in which swollen, painful, chronically inflamed lesions or lumps develop. HS is confined to areas of the body that contain apocrine glands, such as axillac, arcola of the nipple, groin, perincum, circumanal, and periumbilical regions. It is speculated that immunological abnormalities of the hair follicle play a role in the etiology of this disease. HS is a recurring or chronic inflammatory condition affecting particularly young adults, with an average age of onset of 23 years. This poorly understood disease is believed to be under-reported by those who suffer from it, but it is estimated to affect approximately 1% of the general population in the West, with females affected 2 to 5 times more commonly than males (Naldi, L. Epidemiology. In: Hidradenitis Suppurativa; Jemec et al., ed; Heidelberg: Springer. 2006).

HS is characterized by recurrent inflamed nodules, abscesses, and fistulas, and occurs when apocrine gland outlets become blocked by perspiration or are unable to drain normally because of incomplete gland development. Secretions trapped in the glands force perspiration and bacteria into surrounding tissue, causing subcutaneous induration, inflammation, and infection. HS lesions (i.e., nodules, abscesses, and sinuses) are painful and can be malodorous with purulent discharge. This constellation of signs and symptoms results in substantial disability and social stigma for the patients and a profound impact on quality of life.

Current therapies for moderate to severe HS include short- or long-term oral or topical antibiotics, retinoids, intralesional steroids, oral steroids, immunosuppressive agents such as cyclosporine or methotrexate, radiation, laser therapy and tumor necrosis factor-α (TNF-α) antagonist adalimumab. However, adalimumab is the only approved treatment for HS, and other TNF antagonists, such as etanercept, have failed to show improvement of HS over a 24-week treatment period (Adams et al., Arch Dermatol. 146(5): 501-504, 2010). Given the limited success of treatments for HS and the debilitating nature of this disease, there is a pressing need for an effective treatment. Accordingly, it is desirable in the art to provide pediatric patients with safe, well tolerated, and efficacious therapies for treatment of HS.

Pediatric doses (i.e., based on body weight) achieving exposures consistent with efficacy in adult patients are disclosed herein as described above and in Example 1 below. Accordingly, in one aspect is provided a method of treating HS in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of HS in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL

### Solution

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/ml solution.

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet. In some embodiments, the pediatric patient has moderate to severe HS.

In some embodiments, the pediatric patient has had an inadequate response to one or more DMARDs.

In some embodiments, the pediatric patient has had an inadequate response to one or more TNF blockers.

### XII. Treatment of Pediatric Patients with Systemic Lupus Erythematosus

In some embodiments, the pediatric patient has systemic lupus erythematosus (SLE). SLE is an autoimmune disease characterized by antibodies to nuclear and cytoplasmic antigens, multisystem inflammation, protean clinical manifestations, and a relapsing and remitting course. SLE causes widespread inflammation and tissue damage in the affected organs, which may include joints, skin, brain, lungs, kidneys, and blood vessels. Symptoms of SLE vary depending on the affected organs, but may include fatigue, skin rashes, fevers, and pain or swelling in the joints.

Currently, there is no cure for SLE, and existing therapies focus on improving quality of life by controlling symptoms and minimizing flare-ups, primarily through use of immunosuppressive drugs such as hydroxychloroquine and corticosteroids, and immunomodulators such as belimumab and anifrolumab. Despite these treatments, significant unmet therapeutic needs remain for SLE patients. Accordingly, it is desirable in the art to provide pediatric patients with safe, well tolerated, and efficacious therapies for treatment of SLE.

Pediatric doses (i.e., based on body weight) achieving exposures consistent with efficacy in adult patients are disclosed herein as described above and in Example 1 below. Accordingly, in one aspect is provided a method of treating SLE in a pediatric patient utilizing pediatric dosing based on body weight as disclosed herein above. The method generally comprises administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of SLE in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/mL

### Solution

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL

### Solution

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has moderately to severely active SLE.

In some embodiments, the pediatric patient has had an inadequate response to one or more DMARDs.

In some embodiments, the pediatric patient has had an inadequate response to one or more TNF blockers.

### XIII. Treatment of Pediatric Patients with Ulcerative Colitis

In some embodiments, the pediatric patient has ulcerative colitis (UC). UC is one of the two primary forms of idiopathic inflammatory bowel disease (IBD). UC is a chronic, relapsing inflammatory disease of the large intestine characterized by inflammation and ulceration of mainly the mucosal and occasionally submucosal intestinal layers. The hallmark clinical symptoms of UC include bloody diarrhea associated with rectal urgency and tenesmus. The clinical course is marked by exacerbation and remission. Despite the treatment options available to UC patients, significant unmet therapeutic needs remain. Accordingly, it is desirable in the art to provide pediatric patients with safe, well tolerated, and efficacious therapies for treatment of UC.

Pediatric doses achieving exposures consistent with efficacy in adult patients are disclosed herein as described above (i.e., based on body weight). Accordingly, in one aspect is provided a method of treating UC in a pediatric patient utilizing pediatric dosing based on body weight as disclosed herein above. The method involves treating the pediatric patient suffering from UC by administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of UC in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/mL

### Solution

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/ml solution.

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution.

In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet. In some embodiments, the pediatric patient has moderately to severely active UC.

In some embodiments, the pediatric patient has had an inadequate response to one or more DMARDs.

In some embodiments, the pediatric patient has had an inadequate response to one or more TNF blocker.

### XIV. Treatment of Pediatric Patients with Crohn's Disease

In some embodiments, the pediatric patient has Crohn's disease (CD). CD is one of the two primary forms of idiopathic inflammatory bowel disease (IBD). CD is characterized by significant morbidity including abdominal pain, diarrhea, weight loss/malnutrition, fatigue and a progressive nature that leads to complications such as fistulas, strictures and abscesses. Approximately 80% of patients diagnosed with CD will require at least 1 surgery related to the disease at some point in time (Munkholm P, Langholz E, Davidsen M, et al. Gastroenterology. 1993, 105(6): 1716-23). Despite the treatment options available to CD patients, significant unmet therapeutic needs remain. Accordingly, it is desirable in the art to provide pediatric patients with safe, well tolerated, and efficacious therapies for treatment of CD.

Pediatric doses achieving exposures consistent with efficacy in adult patients are disclosed herein as described above (i.e., based on body weight). Accordingly, in one aspect is provided a method of treating UC in a pediatric patient utilizing pediatric dosing based on body weight as disclosed herein above. The method involves treating the pediatric patient suffering from UC by administering upadacitinib to the pediatric patient as a stable oral pharmaceutical formulation or an extended-release tablet. The amount of upadacitinib administered, the oral dose form, and the frequency of dosing (e.g., once or twice daily) will vary based on the weight of the patient.

In some embodiments, the pediatric dosing based on body weight provides exposures consistent with efficacy for the treatment of UC in an adult patient.

In some embodiments, the pediatric patient has a body weight in a range from about 10 to less than about 20 kg, the method comprising administering 3 mg of upadacitinib twice daily (3 mg BID) as an oral solution. In some embodiments, the oral solution comprises upadacitinib at a concentration of about 1 mg/mL, and the 3 mg dose is provided BID as about 3 mL of the about 1 mg/mL solution. In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the oral solution comprises upadacitinib at a concentration of about 1 mg/mL, and the 4 mg dose is provided BID as about 4 mL of the about 1 mg/mL solution. In some embodiments, the oral solution comprises upadacitinib at a concentration of about 0.5 mg/mL and the 4 mg dose is provided BID as about 8 mL of the about 0.5 mg/mL

### Solution

In some embodiments, the pediatric patient has a body weight in a range from about 20 to less than about 30 kg, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 6 mg of upadacitinib twice daily (6 mg BID) as an oral solution. In some embodiments, the oral solution comprises upadacitinib at a concentration of about 1 mg/mL, and the 6 mg dose is provided BID as about 6 mL of the about 1 mg/mL solution. In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 8 mg of upadacitinib twice daily (8 mg BID) as an oral solution. In some embodiments, the oral solution comprises upadacitinib at a concentration of about 1 mg/mL, and the 8 mg dose is provided BID as about 8 mL of the about 1 mg/mL solution. In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 12 mg of upadacitinib twice daily (12 mg BID) as an oral solution. In some embodiments, the oral solution comprises upadacitinib at a concentration of about 1 mg/mL, and the 12 mg dose is provided BID as about 12 mL of the about 1 mg/mL solution. In some embodiments, the oral solution comprises upadacitinib at a concentration of about 0.5 mg/mL and the 12 mg dose is provided BID as about 24 mL of the about 0.5 mg/mL solution.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has a body weight of about 30 kg or greater, the method comprising administering 15 mg of upadacitinib once daily (15 mg QD) as an extended-release tablet. In some embodiments, the pediatric patient has a body weight of about 30 kg or greater, the method comprising administering 30 mg of upadacitinib once daily (30 mg QD) as an extended-release tablet.

In some embodiments, the pediatric patient has moderately to severely active CD.

In some embodiments, the pediatric patient has had an inadequate response to one or more DMARDS.

In some embodiments, the pediatric patient has had an inadequate response to one or more TNF blockers.

### Exemplification

### Example 1: Evaluation of the Pharmacokinetics and Safety of Upadacitinib in Pediatric Subjects with Polyarticular Course Juvenile Idiopathic Arthritis

### Investigational Plan

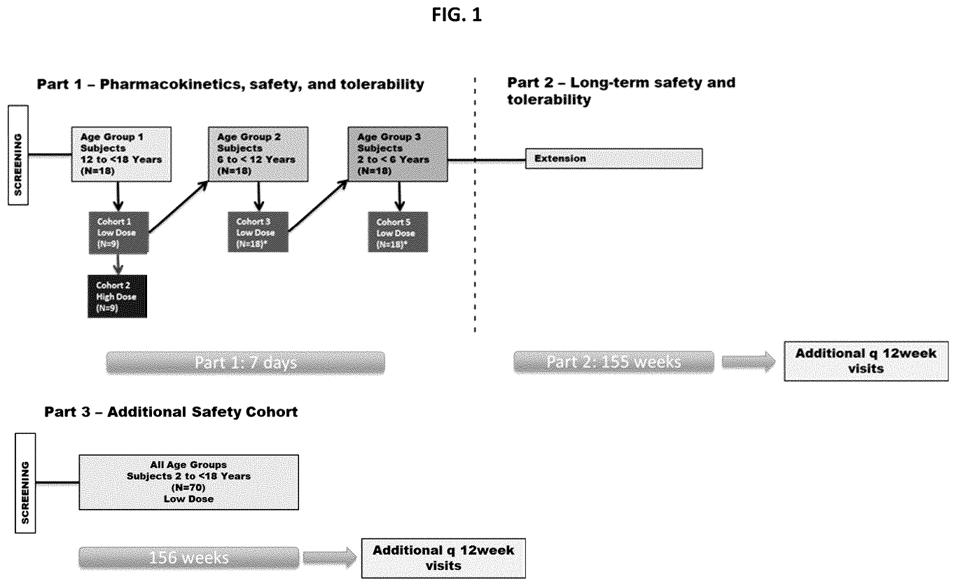

This was a Phase 1, multiple-dose, open-label study consisting of three parts in approximately 124 pediatric subjects ages 2 to less than 18 years with pcJIA. A schematic of the study is shown in FIG. 1. Part 1 was a multiple-dose, open-label, multiple-cohort study. Two sequential multiple-ascending dose levels (Low Dose and High Dose levels) were evaluated in the 12 to <18 years age group (Group 1), and one dose level (Low Dose) was evaluated in the two younger age groups (Group 2: 6 to <12 years, and Group 3: 2 to <6 years). Low Dose and High Dose cohorts in age group 1 enrolled 9 subjects each. Low Dose cohorts in age groups 2 and 3 enrolled approximately 18 subjects each.

Upadacitinib dose was administered based on subject's body weight according to three defined body weight categories per dose level. The study enrolled at least 10 subjects below 30 kg. Recruitment of subjects was conducted through a staggered approach based on age. Subjects who completed Part 1 and were benefiting from study drug with no ongoing adverse events of special interest or serious adverse events, based on investigator's clinical judgment and with subject/family's agreement, had the option to enroll in Part 2 to receive open-label upadacitinib. Part 2 was an open-label, long-term extension to evaluate long-term safety and tolerability of upadacitinib. Subjects in Part 2 received open-label upadacitinib at the equivalent of the Low Dose level per body weight category. At Week 156, if the investigator believed the subject was still benefiting from treatment, they had an option to continue treatment until the end of the study. Part 3 was an additional safety cohort. This additional safety cohort enrolling approximately 70 subjects from all age groups (2 to <18 years) was added to evaluate long-term safety and tolerability of upadacitinib without intensive pharmacokinetic sample collection. Part 3 was open to age groups in which Part I enrollment was completed. Subjects in Part 3 received open-label upadacitinib at the equivalent of the Low Dose level per body weight category, and, after the baseline and screening visits, followed an identical visit schedule as subjects in Part 2, without intensive pharmacokinetic sampling. Doses were adjusted during the study for any of the body weight categories after review of results from the subjects who completed Study Part 1.

### Eligibility Criteria

Male or female subjects, ages 2 to less than 18 years, and total body weight of 10 kg or higher at the time of Screening.
Diagnosed with pcJIA (rheumatoid factor-positive or rheumatoid factor-negative polyarticular JIA, extended oligoarticular JIA, or systemic JIA with active arthritis and without active systemic features) with a history of arthritis affecting at least 5 joints within the first 6 months of disease (for extended oligoarticular JIA: ≤4 joints during the first 6 months of disease and >4 joints thereafter), as per International League of Associations for Rheumatology (ILAR) criteria.
Subject must not have a diagnosis of enthesitis-related arthritis (ERA) or juvenile psoriatic arthritis (JPSA).
Have 5 or more active joints at the time of Screening, defined as the presence of swollen joints (not due to deformity) or, in the absence of swelling, joints with limitation of movement (LOM) plus pain on motion and/or tenderness with palpation, with LOM present in at least three of the active joints.
If receiving methotrexate (MTX), have been taking MTX for at least 12 weeks immediately before and including Study Day 1 on a stable dose of ≤20 mg/m² for at least 8 weeks before and including Study Day 1; in addition, subjects should take either folic acid or folinic acid according to local standard of care.
If on oral glucocorticoids, must have been taking oral glucocorticoids at a stable dose (no greater than 10 mg/day or 0.2 mg/kg/day, whatever is lower) for at least 1 week before and including Study Day 1.
No prior exposure to JAK inhibitor.
No ongoing or active uveitis within 3 months prior to Study Day 1.
Subject has not been treated with intra-articular or parenteral administration of corticosteroids in the preceding 4 weeks prior to Study Day 1.
No history of any malignancy except for successfully treated non-melanoma skin cancer or localized carcinoma in situ of the cervix.
No history of clinically significant (per investigator's judgment) drug or alcohol abuse within the last 6 months.
No history of an allergic reaction or significant sensitivity to constituents of the study drug (and its excipients) and/or other products in the same class.
No current or past history of infection including:
No history of recurrent or disseminated (even a single episode) herpes zoster;
No history of disseminated (even a single episode) herpes simplex;
No human immunodeficiency virus (HIV) infection;
Subject does not have active TB or meet TB exclusionary parameters (specific requirements for TB testing are provided in the Operations Manual);
No active infection(s) requiring treatment with parenteral anti-infectives within 30 days, or oral anti-infectives within 14 days prior to Study Day 1;
No chronic recurring infection and/or active viral infection that, based on the investigator's clinical assessment, makes the subject an unsuitable candidate for the study;
No active hepatitis B virus (HBV) or hepatitis C virus (HCV) infection defined as:
   HBV: hepatitis B surface antigen (HBs Ag) positive (+) or detected sensitivity on the HBV deoxyribonucleic acid (DNA) PCR qualitative test for subjects who are hepatitis B core antibody (HBc Ab) positive (+);
   HCV: HCV ribonucleic acid (RNA) detectable in any subject with anti-HCV antibody (HCV Ab).
Subject has not been a previous recipient of an organ transplant that requires continued immunosuppression.
No history of gastrointestinal perforation (other than appendicitis or penetrating injury), diverticulitis, or significantly increased risk for gastrointestinal perforation per investigator judgment.
No conditions that could interfere with drug absorption including, but not limited to, short bowel syndrome.
No current use of known moderate or strong inhibitors (e.g., amiodarone, clarithromycin, fluconazole, ciprofloxacin, itraconazole, ketoconazole, quinidine, fluoxetine, and paroxetine) or inducers (e.g., carbamazepine, rifampin, phenobarbital, and phenytoin) of drug metabolizing enzymes within 30 days prior to the first dose of study drug through the end of the Part 1 of the study. No systemic use of known strong cytochrome P450 3A isoform subfamily (CYP3A) inhibitors or inducers from the start of Part 2 or Part 3 of the study through study completion.

### Prohibited Medications and Therapy

In addition to the medications listed in the eligibility criteria, the following was NOT allowed:
Prior use of biologic treatment must have been discontinued prior to Study Day 1 (etanercept, 4 weeks; infliximab, adalimumab or abatacept, 8 weeks; golimumab, 10 weeks; tocilizumab, ustekinumab, and certolizumab pegol, 12 weeks; canakinumab, 10 weeks; anakinra, 1 week) and is also prohibited during the study. Note: If there is proper documentation of undetectable drug level measured by a commercially available assay for any of the approved biologics above, there is no minimum washout prior to Baseline.
Immunosuppressant medications must have been discontinued at least 30 days or 5-half-lives prior to study drug administration through the end of the study.
Known current use of moderate or strong inhibitors (e.g., amiodarone, clarithromycin, fluconazole, ciprofloxacin, itraconazole, ketoconazole, quinidine, fluoxetine, and paroxetine) or inducers (e.g., carbamazepine, rifampin, phenobarbital, and phenytoin) of drug metabolizing enzymes within 30 days prior to the first dose of study drug and through the end of Part 1 of the study. Systemic use of known strong CYP3A inhibitors or inducers from the start of Part 2 or Part 3 through the end of the study.
Live vaccines were not permitted during Part 1 of study participation. If the subject and investigator choose to administer live vaccines, these vaccinations were completed, when possible (per local label) at least 4 weeks (8 weeks in Japan) before first dose of study drug with appropriate precautions. Although not mandated by the protocol, vaccines recommended by local guidelines should be considered. If a live vaccine must be administered during Part 2 or Part 3 of study participation, hold study drug for at least 4 weeks (8 weeks in Japan) prior to the vaccination and at least 4 weeks (8 weeks in Japan) after the vaccination. Thereafter, study drug may be resumed at the investigator's discretion with appropriate precautions.
Investigational drugs must have been discontinued within 30 days or 5 half-lives of the drug (whichever is longer) prior to the first dose of study drug. Investigational drugs are also prohibited during the study.
JAK inhibitors (e.g., commercially available upadacitinib [Rinvoq®], tofacitinib [Xeljanz®], ruxolitinib [Jakafi®], baricitinib [Olumiant®], peficitinib [Smyraf®], abrocitinib [PF-04965842], and filgotinib) were prohibited medications during the study.

The following were allowed concomitant medications/therapy:
Stable doses of non-steroidal anti-inflammatory drugs (NSAIDs)
Stable doses of low-dose glucocorticoids (≤0.2 mg/kg/day prednisone; daily maximum, 10 mg)
Stable doses of methotrexate ([MTX]; ≤20 mg/m² body surface area/week)

### Study Drug and Duration of Treatment

The study drug was upadacitinib in the following formats and doses: 7.5 mg tablet, oral; 15 mg tablet, oral; 30 mg tablet, oral; 1 mg/mL solution, oral; 0.5 mg/mL solution, oral. In Part 1, the dose was administered according to three body weight categories. Subjects were categorized based on age group, and the dose administered for seven consecutive days will be based on three different body weight categories per dose level shown in Table 2. The doses evaluated in this study were predicted to provide comparable upadacitinib plasma exposures in each body weight category to those provided by 15 mg QD and 30 mg QD using the extended-release formulation in adult RA subjects. Doses were selected based on population pharmacokinetic analysis of upadacitinib in healthy adults and in adult subjects with RA, and pharmacokinetic simulations across the different body weight categories assuming allometric (weight-based) scaling of upadacitinib pharmacokinetic parameters (volume of distribution and clearance parameters).

The doses selected for this study account for the difference in oral bioavailability between the extended-release tablet formulation and the oral solution. Based on the population pharmacokinetic analyses across Phase 1 through 3 studies, the median (90% prediction interval) upadacitinib average plasma exposures over a dosing interval are 15.1 ng/ml (8.96 ng/mL to 32.7 ng/mL) for 15 mg QD and 30.0 ng/mL (18.1 ng/ml to 63.8 ng/ml) for 30 mg QD using the extended-release formulation in adult RA patients; similar exposures were predicted to be achieved in pediatric patients by the low and high doses, respectively, within each body weight category based on pharmacokinetic simulations (Table 2). Doses were adjusted during the study for any of the body weight categories after review of results from the subjects who completed Part 1.

TABLE 2
Upadacitinib Dosing by Body Weight Category and Dose Level

| | Body Weight Category* | | |
|---|---|---|---|
| Dose | 10 to | 20 to | |
| Level | <20 kg | <30 kg | ≥30 kg |
| Low | 3 mg | 4 mg | 15 mg QD Tablet |
| Dose | BID Oral | BID Oral | (or 6 mg BID Oral |
| | Solution | Solution | Solution if unable |
| | | | to swallow tablet) |
| High | 6 mg | 8 mg | 30 mg QD Tablet |
| Dose | BID Oral | BID Oral | (or 12 mg BID Oral |
| | Solution | Solution | Solution if unable |
| | | | to swallow tablet) |

BID = twice daily;
QD = once daily

Subjects in Part 2 received open-label upadacitinib at the Low Dose level described in Table 2. At Week 156, if the investigator believed the subject was still benefiting from treatment, they had the option to continue treatment at the equivalent of the Low Dose level per body weight category until the end of the study.

### Dose Adjustment Criteria

For each subject, upadacitinib dose in Part I was based on the subject's body weight at Screening and the assigned study cohort. When preliminary pharmacokinetic and clinical data (following Part 1 Day 7 intensive pharmacokinetic sampling) from at least 4 subjects was available in a cohort, these data were used to adjust doses, if needed, for subjects who were enrolled (including subjects in Parts 2 and 3) and for subjects who were subsequently enrolled. Additional criteria were considered for making dose adjustment decisions for Part 1 to ensure safe and efficacious exposures.

Subjects who were receiving upadacitinib in Part 2 continued to receive the same dose until their next scheduled visit (unless earlier dose adjustment was deemed necessary by the investigator). On the next scheduled visit, subjects in Part 2 received upadacitinib according to the dosing regimen (Table 2) for the remaining duration of the study. Subjects weighing >30 kg had the option to receive upadacitinib oral solution if unable to swallow the tablet (see Table 2). For subjects in Part 2 who underwent dose adjustments, blood samples for upadacitinib pharmacokinetics were collected prior to dosing and at 1 hour and 2 hours after dosing from subjects at an unscheduled visit that was 4-6 weeks after the subject's dose adjustment visit. The timing of the last two doses administered prior to the pre-dose trough pharmacokinetic sample and the timing of the blood sample collections were recorded to the nearest minute. At the same unscheduled visit, blood samples for clinical laboratory testing were also collected.

Subjects in Parts 2 and 3 were dosed according to the Low Dose level per body weight category (Table 2) or as determined based on data available from Part 1. A change in the subject's body weight category was determined by the investigator at any of the study visits in Parts 2 and 3. A pharmacokinetic blood sample was collected in subjects in Parts 2 and 3 when dose was modified due to change in the subjects' body weight.

### Study Objectives and Endpoints

### Part 1:

To evaluate the pharmacokinetics, safety, and tolerability of multiple doses of upadacitinib in pediatric subjects with pcJIA. To evaluate the palatability of upadacitinib oral solution in pediatric subjects. To evaluate descriptive efficacy of upadacitinib in pcJIA.

### Part 2:

To evaluate the long-term safety and tolerability of upadacitinib in pediatric subjects with pcJIA who completed Part 1. To evaluate descriptive efficacy of upadacitinib in pcJIA.

### Part 3:

To evaluate the long-term safety and tolerability of upadacitinib in pediatric subjects with pcJIA. To evaluate descriptive efficacy of upadacitinib in pcJIA.

During Part 2 and Part 3, subjects who did not achieve at least 20% improvement in total number of active joints (joints with swelling not due to deformity or joints with LOM and with pain, tenderness, or both) compared to baseline at 2 consecutive visits on or after Week 8 discontinued study drug and received treatment at the investigator's discretion in accordance with local standard-of-care.

### Safety Endpoints

Safety evaluations included incidence of Treatment Emergent Adverse Events (TEAEs), physical examination results, change in vital sign measurements, and clinical laboratory testing (hematology and chemistry) as measures of safety and tolerability for the entire study duration.

### Pharmacokinetic Endpoints

For Part 1, the values for the pharmacokinetic parameters of upadacitinib including Cₘₐₓ, time to maximum observed plasma concentration (Tₘₐₓ), area under the plasma concentration versus time curve during a dosing interval (AUCₜₐᵤ) on Day 7, apparent oral clearance at steady state (CL/F), and half-life were determined using non-compartmental methods.

### Efficacy Endpoints

The following efficacy parameters were used to determine JIA American College of Rheumatology (ACR) response and Juvenile Arthritis Disease Activity Score (JADAS) were collected:
Total number of active joints defined as:
   joints with swelling not due to deformity, OR
   joints with limitation of movement [LOM] with pain, tenderness or both
Number of joints with LOM
Childhood Health Assessment Questionnaire (C-HAQ)
Physician's Global Assessment of Disease Activity (visual analog scale [VAS])
Patient's/Caregiver's Global Assessment of Overall Well Being (VAS)
Erythrocyte sedimentation rate (ESR)
C-reactive protein (CRP)

Based on these parameters, the following composite efficacy endpoints were evaluated:
JIA ACR pediatric 30/50/70/90/100 responses
Change from Baseline in JADAS10/27/71 responses, as well as JADAS-based criteria for low disease activity and remission (if deemed useful and appropriate).

### Example 2: Pharmacokinetics of Upadacitinib in Pediatric Patients with Polyarticular Course Juvenile Idiopathic Arthritis (Pc.JIA); Analysis of Interim Results of Example 1

### Objective

The primary objective of this analysis was to characterize the pharmacokinetics from a Phase 1 study of upadacitinib in children with pcJIA (PK analysis of Example 1).

### Method

Patients (N=51) diagnosed with pcJIA were enrolled into one of four groups in an open-label, multiple-dose study (Group 1, 12 to <18 years, low dose; Group 2, 12 to <18 years, high dose; Group 3, 6 to <12 years, low dose; Group 4, 2 to <6 years, low dose). The low and high doses were selected to provide comparable plasma exposures in pediatrics to 15 mg and 30 mg QD doses of ER tablet formulation in adults, respectively. Patients received bodyweight-based upadacitinib doses either as twice-daily (BID) immediate-release (IR) oral solution or QD extended-release (ER) tablet formulation. Pharmacokinetic assessment was performed at steady state on Study Day 7, after which all patients might continue the study with low dose.

### Results

A summary of the demographics of the enrolled subjects is provided in Table 3. Pharmacokinetic results from 49 patients with evaluable drug concentrations on Study Day 7 were reported.

In Group 1, the geometric mean upadacitinib maximum plasma concentration (Cₘₐₓ) and AUC₀₋₂₄ at steady state were 35.1 ng/ml and 269 ng·h/mL, respectively.

In Group 2, the geometric mean upadacitinib Cₘₐₓ and AUC₀₋₂₄ were 69.8 ng/ml and 553 ng·h/mL, respectively.

In Group 3, the geometric mean upadacitinib Cₘₐₓ and AUC₀₋₂₄ were 51.0 ng/ml and 346 ng·h/mL, respectively.

In Group 4, the geometric mean upadacitinib Cₘₐₓ and AUC₀₋₂₄ were 46.6 ng/mL and 369 ng·h/mL, respectively.

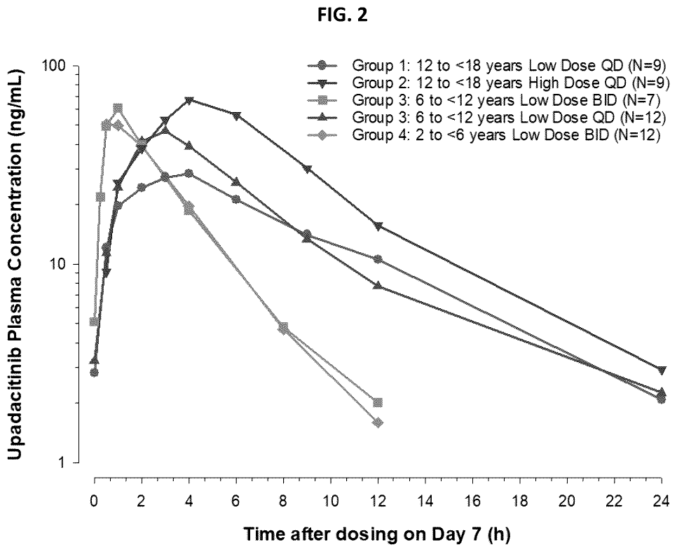

The median time to maximum upadacitinib concentration was approximately 3 hours and 1 hour; and the harmonic mean functional half-life was approximately 5 hours and 2 hours for the QD ER tablet and the BID IR solution regimens, respectively. Upadacitinib apparent oral clearance increased in pcJIA patients with increasing bodyweight. The mean upadacitinib plasma concentration-time profiles of each group are presented by dosing regimen in FIG. 2. Overall, the data indicate that upadacitinib plasma exposures in pediatric patients with pcJIA were comparable to target adult RA patients at the evaluated dosing regimens with the ER tablet or IR solution and at both low and high dose levels. These results support the use of a bodyweight-based dosing regimen in upadacitinib pediatric studies.

TABLE 3
Demographics Summary of Enrolled Pediatric pcJIA Patients

| | | Group 1 | Group 2 | Group 3 | Group 4 |
|---|---|---|---|---|---|
| | | (N = 9) | (N = 9) | (N = 19) | (N = 14^{a}) |
| Age | Mean ± SD | 14.9 ± 1.5 | 13.9 ± 1.2 | 9.5 ± 1.6 | 3.6 ± 1.5 |
| (years) | (Range) | (13-17) | (12-16) | (6-12) | (2-6) |
| Weight | Mean ± SD | 61.3 ± 14.6 | 51.7 ± 13.0 | 37.8 ± 14.4 | 15.1 ± 3.2 |
| (kg) | (Range) | (42.0-92.9) | (32.5-71.5) | (19.7 ± 72.1) | (11.0 ± 22.5) |
| Sex | Male, N (%) | 2 (22.2) | 1 (11.1) | 4 (21.1) | 3 (21.4) |
| | Female, N (%) | 7 (77.8) | 8 (88.9) | 15 (78.9) | 11 (78.6) |
| Dose | Oral Solution, | 0 (0.0) | 0 (0.0) | 7 (36.8) | 14 (100.0) |
| Form | N (%) | | | | |
| | Tablet, N (%) | 9 (100.0) | 9 (100.0) | 12 (63.2) | 0 (0.0) |

^{a}Two subjects were excluded from pharmacokinetic analysis. One subject mistakenly received half dose and the other subject had incomplete PK sample collection.

### Example 3: Safety and Efficacy of Upadacitinib for Pediatric Patients with Polyarticular Course Juvenile Idiopathic Arthritis: An Interim Analysis of an Open-Label, Phase 1 Trial (Analysis of Interim Results of Example 1 Through Week 12)

### Objective

The objective of this study was to evaluate the safety and efficacy of upadacitinib in pediatric patients with pcJIA by age groups.

### Method

This open-label, 3-part, Phase 1 trial (NCT03725007) enrolled pediatric patients aged 2 to <18 years with pcJIA and >5 active joints at 31 sites across North America, Europe, and Asia In part 1, two sequential multiple ascending UPA dose groups (low: 3 mg or 4 mg twice-daily oral solution, or 15 mg once-daily tablet; high: 6 mg or 8 mg oral solution, or 30 mg tablet) were administered based on body weight groups (10 to <20, 20 to <30, and ≥30 kg) and age groups (2 to <6, 6 to <12, and 12 to <18 years of age) for 7 days. In part 2 (long-term extension of part 1) and part 3 (additional safety cohort), low-dose UPA (3 mg or 4 mg oral solution, or 15 mg tablet) was administered based on body weight groups for up to 156 weeks. Efficacy endpoints included the American College of Rheumatology (ACR) response 30, 50, and 70; the Childhood Health Assessment Questionnaire (C-HAQ); and the 27-point Juvenile Arthritis Disease Activity Score (JADAS-27) at week 12 among patients treated in part 1 and part 2. This was an interim analysis.

### Results

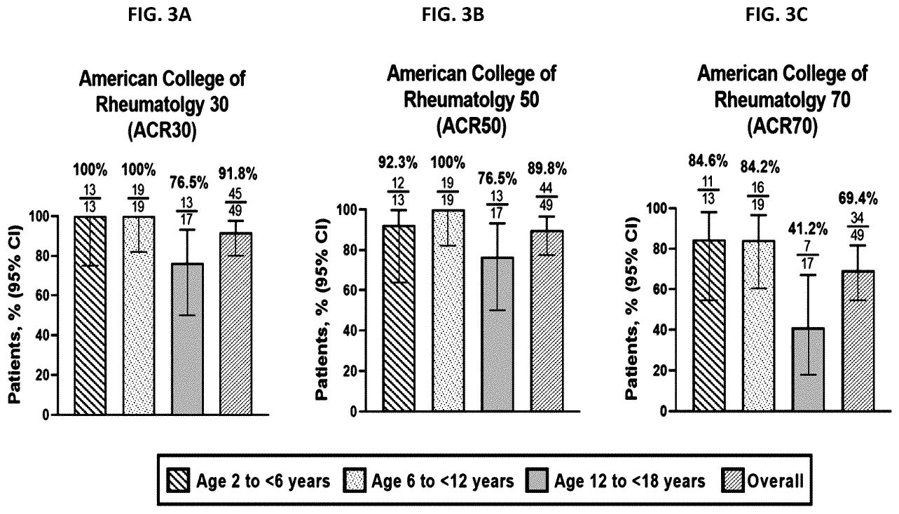

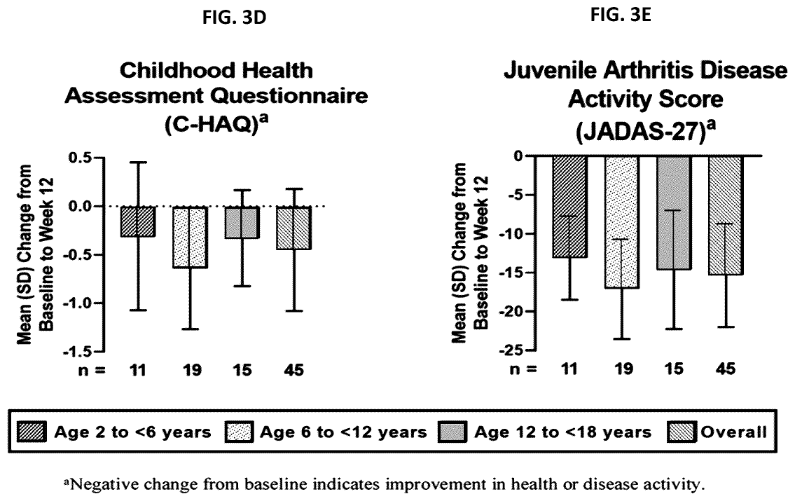

A total of 57 pediatric patients with mean (SD) age of 9.5 (4.4) years, 78.9% female, and mean (SD) weight of 38.1 (20.4) kg received UPA. In part 1, 8 (15.7%) out of 51 patients reported adverse events (AEs) through 7 days; no patients reported serious AEs or AEs leading to treatment discontinuation. In part 2 and part 3, 52 (91.2%) out of 57 patients reported AEs that were predominately mild to moderate in severity (Table 4). Rates of AEs were generally highest among patients 12 to <18 years of age. The most common AEs of special interest included elevated creatine phosphokinase (n=6/57, 10.5%), hepatic disorder (n=3/57, 5.3%), and neutropenia (n=2/57, 3.5%). Six (31.6%) out of 19 patients in the 12 to <18 years of age group reported serious AEs and 2 (10.5%) reported AEs leading to treatment discontinuation. No deaths occurred. A high proportion of patients across all age groups achieved ACR30, ACR50, and ACR70 response at week 12 (FIGS. 3A, 3B, and 3C, respectively). Referring to FIGS. 3A-3C, numbers above the bars are the proportion of patients with response and (n/N). Improvement from baseline to week 12 in C-HAQ and JADAS-27 scores was observed across all age groups (FIGS. 3D and 3E, respectively). Overall, in pediatric patients with pcJIA, upadacitinib was safe and well tolerated and associated with improvements in disease activity in a high proportion of patients at week 12 of this interim analysis.

TABLE 4
Interim Analysis of Safety of Upadacitinib up to 156 Weeks

| | Age | Age | Age | |
|---|---|---|---|---|
| | 2 to <6 | 6 to <12 | 12 to <18 | |
| | years | years | years | Total |
| AE, n (%) | n = 14 | n = 24 | n = 19 | N = 57 |
| Any AE | 11 (78.6) | 22 (91.7) | 19 (100) | 52 (91.2) |
| Any serious AE | 0 | 0 | 6 (31.6) | 6 (10.5) |
| AE leading to | 0 | 0 | 2 (10.5) | 2 (3.5) |
| discontinuation | | | | |
| of study drug | | | | |
| Deaths | 0 | 0 | 0 | 0 |
| AEs of special interest | | | | |
| Serious infections | 0 | 0 | 1 (5.3) | 1 (1.8) |
| Opportunistic infection | 0 | 0 | 1 (5.3) | 1 (1.8) |
| Hepatic disorder | 0 | 2 (8.3) | 1 (5.3) | 3 (5.3) |
| Anemia | 1 (7.1) | 0 | 0 | 1 (1.8) |
| Neutropenia | 0 | 2 (8.3) | 0 | 2 (3.5) |
| Lymphopenia | 0 | 0 | 1 (5.3) | 1 (1.8) |
| Elevated creatine | 0 | 3 (12.5) | 3 (15.8) | 6 (10.5) |
| phosphokinase (CPK) | | | | |

### Example 4: Pharmacokinetics Safety, and Tolerability of Upadacitinib in Children with Atopic Dermatitis

The objective of this study was to characterize the pharmacokinetics (PK), safety, and tolerability of upadacitinib in children with severe atopic dermatitis (AD).

### Method

This was an open-label, multiple-dose study. AD patients (N=35) were enrolled into four cohorts (Cohort 1, 6 to <12 years with low dose; Cohort 2, 6 to <12 years with high dose; Cohort 3, 2 to <6 years with low dose; Cohort 4, 2 to <6 years with high dose). Upadacitinib was administered based on body weight with either BID oral solution or QD extended-release tablet. The low and high doses were selected to provide comparable plasma exposure in pediatric patients to 15 mg and 30 mg QD doses in adults, respectively. PK was evaluated on Day 7 post first dose. Safety was evaluated throughout the study. Exploratory efficacy parameters were collected at specified timepoints.

### Results

Geometric mean Cₘₐₓ and AUC over 0-24 hours at steady-state were 33.1 and 35.2 ng/ml and 249 and 264 ng·h/mL in Cohorts 1 and 3; 95.5 and 101 ng/ml and 523 and 625 ng·h/mL in Cohorts 2 and 4, respectively.

Upadacitinib was generally safe and well tolerated. The most common AEs were COVID infection, headache, and abdominal discomfort. No new safety risks were identified compared to the known safety profile for upadacitinib.

In the 29 subjects with available interim efficacy results at week 12, 34.5% achieved validated Investigator's Global Assessment scale for AD score of 0 or 1 and 69.0% achieved Eczema Area and Severity Index by at least 75% at Week 12 with treatment of upadacitinib. Overall, the findings support the use of a bodyweight-based dosing regimen for further investigation of upadacitinib in upcoming Phase 3 clinical trials in pediatric AD patients.

### Example 5: Upadacitinib for the Treatment of Severe Atopic Dermatitis in Pediatric Subjects

This study was an open-label, multiple-dose phase 1 study to evaluate the pharmacokinetic (PK), safety, and tolerability of upadacitinib in pediatric subjects with severe atopic dermatitis (AD), which remains ongoing.

### Study Design

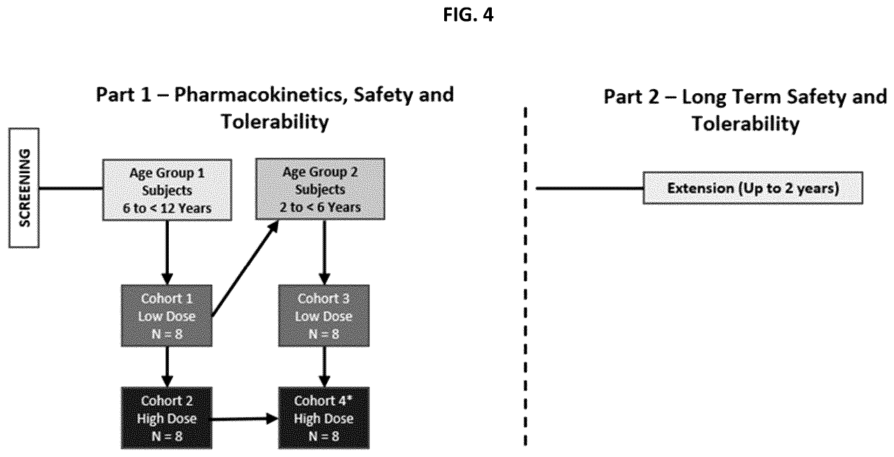

A schematic of the study design is provided in FIG. 4. With reference to FIG. 4, the study consisted of two parts.

Part 1 was a multiple-dose, open-label, multiple-cohort study that consisted of two sequential multiple-ascending dose groups (Low Dose and High Dose levels) in the two age groups. (Group 1: 6 to <12 years and Group 2: 2 to <6 years). Upadacitinib dose was administered based on subject's body weight according to pre-defined body weight categories per dose level with at least 4 subjects in each body weight category (Table 5). The objectives of Part 1 were to evaluate the pharmacokinetics, activity, safety, and tolerability of multiple doses of upadacitinib in pediatric subjects with severe atopic dermatitis and to evaluate the palatability of upadacitinib oral solution in pediatric subjects.

Part 2 was a multiple-dose, open-label, long-term extension to evaluate long term safety and tolerability of upadacitinib. Subjects in Part 2 received open-label upadacitinib at the equivalent of the Low Dose level per the body weight category.

TABLE 5
Upadacitinib Dosing by Body Weight Category
and Dose Level for Subjects 2 to <12 years

| | Body Weight Category* | | |
|---|---|---|---|
| Dose | Current Dosing Scheme Starting with Protocol Version 4.0 | | |
| Level | 10 to <20 kg | 20 to <30 kg | ≥30 kg |
| Low | 3 mg BID | 4 mg BID | 15 mg QD Tablet |
| Dose | Oral Solution | Oral Solution | (Or 6 mg BID Oral |
| | | | Solution if unable |
| | | | to swallow tablet) |
| High | 6 mg BID | 8 mg BID | 30 mg QD Tablet |
| Dose | Oral Solution | Oral Solution | (Or 12 mg BID Oral |
| | | | Solution if unable |
| | | | to swallow tablet) |

| Dose | Previous Dosing Scheme Before Protocol Version 4.0 | | | |
|---|---|---|---|---|
| Level | 10 to <15 kg | 15 to <25 kg | 25 to <40 kg | ≥40 kg |
| Low | 1.6 mg BID IR | 2 mg BID IR | 7.5 mg QD ER | 15 mg QD ER |
| Dose | Oral Solution | Oral Solution | Tablet or | Tablet or |
| | | | 3 mg BID IR | 6 mg BID IR |
| | | | Oral Solution | Oral Solution |
| High | 3.2 mg BID IR | 4 mg BID IR | 15 mg QD ER | 30 mg QD ER |
| Dose | Oral Solution | Oral Solution | Tablet or | Tablet or |
| | | | 6 mg BID IR | 12 mg BID IR |
| | | | Oral Solution | Oral Solution |

BID = twice daily;
QD = once daily
*At least 4 children will be enrolled in each weight category.

Subjects who completed Part 1 had the option to enroll in Part 2 and receive open-label low dose upadacitinib. During Part 2 of the study, concomitant topical medications for AD were allowed during the study per investigator discretion. Subjects with an Eczema Area Severity Index (EASI) score worsening of 25% or more compared with their Baseline EASI score at any 2 consecutive scheduled study visits after Week 4 or subjects who did not achieve at least a 50% improvement in their EASI score compared to baseline at 2 consecutive visits on or after Week 8 discontinued study drug and received treatment at investigator's discretion and in accordance with local standard-of-care.

### Key Eligibility Criteria

Male or female subjects, ages 2 years to less than 12 years at Screening, and total body weight 10 kg or higher at the time of baseline were eligible to enroll. Criteria for AD included:
Subject diagnosed with AD with onset of symptoms at least 6 months prior to baseline
Subject meets Hanifin and Rajka criteria for AD.
Active disease as defined by the following disease activity criteria at the Screening and Baseline Visits (all must be met):
   Eczema Area and Severity Index score ≥21;
   Validated Investigator Global Assessment for AD (vIGA-AD) score equal to 4;
   ≥15% body surface area of AD involvement
Have documented history (within 12 months prior to the Baseline Visit) of inadequate response or intolerance to topical corticosteroids (TCS) and topical calcineurin inhibitor (TCI) OR for whom use of TCS and TCIs is otherwise medically inadvisable.

### Study Population

Between 31 Jan. 2019 and 18 Mar. 2022, a total of 32 subjects enrolled in the study and received at least one dose of upadacitinib. Enrollment into Cohort 4 is ongoing. An additional 20 subjects were screen failures. The most common reason for pre-screen failure was the inability of subjects to meet the Hanifin and Rayka criteria for AD. One subject (3.1%) and 10 subjects (32.3%) in Part 1 and Part 2, respectively, discontinued upadacitinib. In Part 2, the most common reason for discontinuation was lack of efficacy (n=4, 12.9%) and adverse event (n=3, 9.7%).

The median duration of upadacitinib exposure across all study subjects in Part 1 was 7.0 days (range: 1 to 9 days) and 170 days (range: 1 to 770 days) in Part 2 (Table 6).

TABLE 6
Duration of Exposure
Median duration of exposure, days (range)
Part 1

| Cohort 1 | Cohort 2 | Cohort 3 | Cohort 4 | Total |
|---|---|---|---|---|
| (N = 9) | (N = 8) | (N = 9) | (N = 6) | (N = 32) |
| 7.0 | 7.0 | 7.0 | 7.0 | 7.0 |
| (7-7) | (7-7) | (1-9) | (7-7) | (1-9) |

Part 2

| Aged 6 to <12 years | Aged 2 to < 6 years | Total |
|---|---|---|
| (N = 17) | (N = 14) | (N = 31) |
| 425 | 23.0 | 170 |
| (1-770) | (1-623) | (1-770) |

Demographics (Table 7) and baseline disease characteristics (Table 8) for all subjects enrolled are summarized. The majority of subjects who received upadacitinib were female (n=18, 56.3%), White (n=19, 59.4%), and the median age was 6.0 years (range: 2 to 11 years).

### Preliminary Clinical Pharmacokinetics

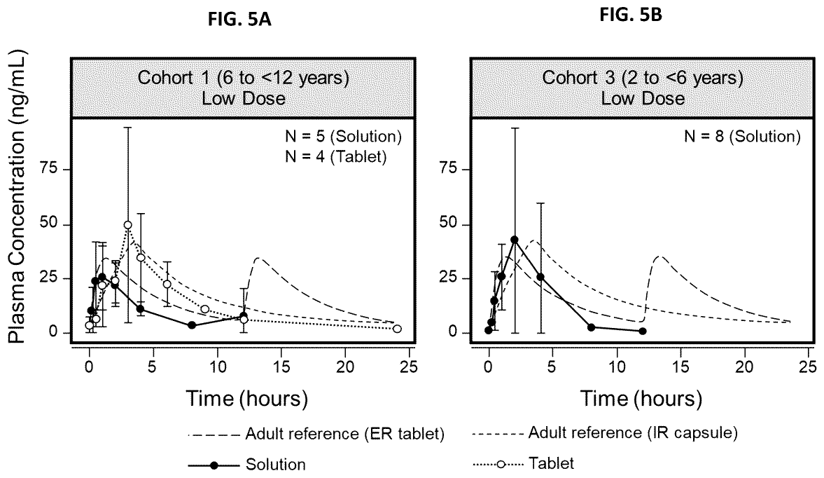

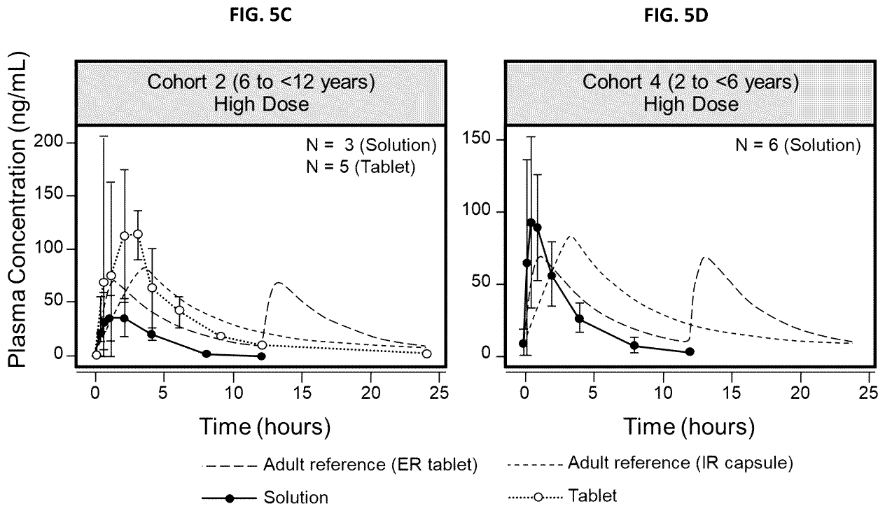

A summary of upadacitinib PK parameters on Day 7 are provided in FIGS. 5A, 5B, 5C, and 5D and Table 9. For Cohorts 1, 2, 3 and 4, there were 9, 8, 6, and 0 subjects who received upadacitinib at the original protocol defined dosing regimen and 0, 0, 2 and 6 subjects received upadacitinib at the revised dosing regimen. There was one additional subject who was enrolled in Cohort 3 but withdrew informed consent before PK assessment. This subject was therefore not included in PK analyses. For the analyses of plasma concentration versus time profiles and pharmacokinetic parameters, data from all these subjects were summarized based on cohort and dosage form (FIGS. 5A-5D and Table 9).

### Preliminary Clinical Efficacy

Clinical efficacy parameters were collected as exploratory measures and to facilitate the assessment of benefit-risk to justify a continuation of Low Dose upadacitinib during Part 2, the long-term safety and tolerability portion of the study. No formal statistical comparisons were planned. Since all cohorts received Low Dose upadacitinib in Part 2, the overall population was summarized for efficacy.

The pharmacodynamic activity of upadacitinib was demonstrated by efficacy outcome measures that evaluated improvements from baseline across multiple study timepoints. A validated Investigator's Global Assessment scale for Atopic Dermatitis (vIGA-AD) score of 0 or 1 (with at least two grades of reduction from baseline) at Week 12 was achieved by 9/27 subjects (33.3%) in the overall population across cohorts as of the data cutoff (Table 10), signifying achievement of clear or almost clear skin. Notably, activity was observed in the overall population despite 10 subjects in Cohorts 1, 2, and 3 requiring a dose increase to adequately reach the target upadacitinib plasma exposure.

Change from baseline of the Eczema Arca and Severity Index by at least 75% (EASI 75) at Week 12 was achieved by 19/27 subjects (70.4%) for the overall population across cohorts as of the data cutoff (Table 11). The mean and median percent change from baseline of the EASI score at Week 12 for the overall population across cohorts was −79.8% and −82.9%, respectively as of the data cutoff (Table 12). For Table 10 to Table 12, results are listed for the overall population, the cohort of 2 to <6 years of age, the cohort of 6 to <12 years of age, subjects enrolled under the original dosing scheme, and subjects enrolled after the dosing scheme was readjusted to reach the target upadacitinib plasma exposure.

Efficacy outcomes are also summarized for the subgroup of 8 subjects from Cohorts 3 and 4, who were treated with a revised upadacitinib dosing scheme from study entry. Although efficacy outcomes from only 7 of these subjects are available at Week 12, all subjects achieved an EASI 75 response at Week 12. The outcomes of EASI 75, vIGA-AD 0/1 and percent change from baseline of the EASI score in these subjects were consistent with those in the overall population at respective timepoints. For those 10 subjects enrolled under protocol version 3.0 or earlier whose dose was increased at different points in time due to the dose modification implemented with protocol v4.0, response rates as measured by EASI75 and vIGA-AD score of 0 or 1 (with at least two grades of reduction from baseline) numerically improved 12 weeks after the dose increase.

TABLE 7
Demographics

| | Cohort 1 | Cohort 2 | Cohort 3 | Cohort 4 | Total |
|---|---|---|---|---|---|
| | (N = 9) | (N = 8) | (N = 9) | (N = 6) | (N = 32) |
| Sex, n (%) | | | | | |
| Male | 3 (33.3) | 2 (25.0) | 4 (44.4) | 5.1 (83.3) | 14 (43.8) |
| Female | 6 (66.7) | 6 (75.0) | 5 (55.6) | 1 (16.7) | 18 (56.3) |
| Age, years, median (range) | 8.0 (6-11) | 7.5 (6-10) | 4.0 (2-5) | 3.0 (2-4) | 6.0 (2-11) |
| Race, n (%) | | | | | |
| White | | | | | |
| Black or African American | 4 (44.4) | 6 (75.0) | 6 (66.7) | 3 (50.0) | 19 (59.4) |
| Asian | 3 (33.3) | 1 (12.5 | 2 (22.2) | 2 (33.3) | 8 (25.0) |
| American Indian/Alaska | 1 (11.1) | 1 (12.5) | 1 (11.1) | 0 | 3 (9.4) |
| Native | 0 | 0 | 0 | 0 | 0 |
| Native Hawaiian or Other | 0 | 0 | 0 | 0 | 0 |
| Pacific Islander | 0 | 0 | 0 | 0 | 0 |
| Other | 1 (11.1) | 0 | 0 | 1 (16.7) | 2 (6.3) |
| Multiple | | | | | |
| Ethnicity, n (%) | | | | | |
| Hispanic or Latino | 4 (44.4) | 2 (25.0) | 2 (22.2) | 0 | 8 (25.0) |
| Not Hispanic or Latino | 5 (55.6) | 6 (75.0) | 7 (77.8) | 6 (100) | 24 (75.0) |
| Weight, kg, median (range) | 26.8 (20.7-45.0) | 33.7 (20.4-56.6) | 18.8 (13.1-26.2) | 15.2 (13.7-18.4) | 22.0 (13.1-56.6) |
| BMI, kg/m2, median (range) | 15.9 (14.5-23.6) | 21.1 (14.4-32.7) | 16.0 (14.6-24.9) | 16.2 (14.2-20.6) | 16.2 (14.2-32.7) |

TABLE 8
Baseline Disease Characteristics

| | Cohort 1 | Cohort 2 | Cohort 3 | Cohort 4 | Total |
|---|---|---|---|---|---|
| | (N = 9) | (N = 8) | (N = 9) | (N = 6) | (N = 32) |
| Baseline vIGA-AD, n (%) | | | | | |
| <4 | 0 | 0 | 0 | 0 | 0 |
| 4 | 9 (100) | 8 (100) | 9 (100) | 6(100) | 32 (100) |
| EASI, median (range) | 27.4 (22.0-59.7) | 31.6 (23.4-53.0) | 28.8 (21.6-47.0) | 28.5 (24.3-32.7) | 28.8 (21.6-59.7) |
| BSA (%) | 50.0 (20-98) | 40.5 (28-100) | 44.0 (25-76) | 56.0 (45-80) | 50.0 (20-100) |
| Disease history, n (%) | | | | | |
| Yes | 5 (55.6) | 1(12.5) | 3 (33.3) | 1 (16.7) | 10 (31.3) |
| No | 4 (44.4) | 7 (87.5) | 6 (66.7) | 5 (83.3) | 22 (68.8) |

BSA = body surface area
EASI = Eczema Area and Severity Index

TABLE 9
Summary of Pharmacokinetic Parameters of Upadacitinib
in Pediatric Subjects with AD

| Pharmacokinetic | 6 to < 12 years | | 2 to < 6 years |
|---|---|---|---|
| Parameter | (N=17) | | (N = 13) |
| | Cohort 1 | | Cohort 3 |
| | IR Solution | ER Tablet | IR Solution |
| Low Dose | (N = 5) | (N = 4) | (N = 7) |
| Cₘₐₓ (ng/mL) | 24.7 (27.9, 53) | 47.7 (57.2, 72) | 35.2 (47.4, 103) |
| Tₘₐₓ^{a} (h) | 1.0 (0.5, 2.0) | 2.5 (1.0, 4.0) | 2.0 (1.0, 4.0) |
| t_{1/2}^{b} (h) | 2.35 (0.59) | 5.79 (1.20) | 1.67 (0.66) |
| AUC₀₋₂₄^{c} | 224 (248, 55) | 283 (289, 23) | 264 (349, 107) |
| (ng · h/mL) | | | |
| CLₛₛ/F (L/h) | 19.3 (20.2, 31) | 31.5 (34.2, 48) | 15.6 (18.6, 52) |
| | Cohort 2 | | Cohort 4 |
| | IR Solution | ER Tablet | IR Solution |
| High Dose | (N = 3) | (N = 5) | (N = 6) |
| Cₘₐₓ (ng/mL) | 43.2 (46.8, 43) | 154 (166, 50) | 96.9 (111, 51) |
| Tₘₐₓ^{a} (h) | 2.0 (0.25, 4.0) | 2.0 (0.5, 3.0) | 0.75 (0.25, 1.0) |
| t_{1/2}^{b} (h) | 1.68 (0.34) | 4.93 (1.06) | 2.25 (0.514) |
| AUC₀₋₂₄^{c} | 347 (355, 25) | 668 (679, 20) | 579 (633, 38) |
| (ng · h/mL) | | | |
| CLₛₛ/F (L/h) | 23.0 (23.6, 27) | 39.1 (42.3, 37) | 20.7 (23.6, 66) |

AUC₀₋₂₄ = area under the plasma concentration-time curve over the last 24 hr dosing interval;
BID = twice daily;
Cₘₐₓ = maximal concentration;
CLₛₛ/F = apparent total body clearance after oral administration (at steady state);
ER = extended release;
IR = immediate release;
QD = once daily;
t_{1/2} = half-life;
Tₘₐₓ = time to maximal concentration
^{a}Median (min-max).
^{b}Harmonic mean (pseudo standard deviation); half-life within a dosing interval.
^{c}AUC₀₋₂₄ for solution formulated was calculated as AUC₀₋₁₂ × 2.
Note:
Parameters are expressed as geometric mean (mean, % coefficient of variation) unless otherwise noted.

TABLE 10
Proportion of Subjects Achieving vIGA-AD of 0 or 1 with at Least
Two Grades of Reduction from Baseline at Week 12 (As Observed)

| | N | Responder, n (%) | 95% CI |
|---|---|---|---|
| Week 12 | | | |
| Overall | 27 | 9 (33.3) | 16.5-54.0 |
| Age 2 to <6 years | 12 | 3 (25.0) | 5.5-57.2 |
| Age 6 to <12 years | 15 | 6 (40.0) | 16.3-67.7 |
| Enrollment up to protocol v3.0 | 20 | 6 (30.0) | 11.9-54.3 |
| Enrollment on or after protocol v4.0 | 7 | 3 (42.9) | 9.9-81.6 |

Exact confidence intervals based on Clopper-Pearson method.

TABLE 11
Proportion of Subjects Achieving EASI 75 at Week
12 (As Observed) (Intention-to-Treat Population)

| | | Responder, | |
|---|---|---|---|
| | N | n (%) | 95% CI |
| Week 12 | | | |
| Overall | 27 | 19 (70.4) | 49.8-86.2 |
| Age 2 to <6 years | 12 | 9 (75.0) | 42.8-94.5 |
| Age 6 to <12 years | 15 | 10 (66.7) | 38.4-88.2 |
| Enrollment up to protocol v3.0 | 20 | 12 (60.0) | 36.1-80.9 |
| Enrollment on or after protocol v4.0 | 7 | 7 (100) | 59.0-100.0 |

Exact confidence intervals based on Clopper-Pearson method.

TABLE 12
Change and Percent Change in EASI at Week 12
(As Observed) (Intention-to-Treat Population)

| | | Change in EASI | | Percent Change in EASI | |
|---|---|---|---|---|---|
| | N | Mean | Median | Mean | Median |
| Week 12 | | | | | |
| Overall | 27 | −24.9 | −23.8 | −79.8 | −82.9 |
| | | (9.61) | (−29.0, −18.2) | (19.0) | (−93.0, −69.4) |
| Age 2 to <6 | 12 | −24.8 | −25.2 | −82.0 | −82.8 |
| years | | (6.27) | (−26.8, −20.4) | (14.6) | (−92.5, −73.3) |
| Age 6 to <12 | 15 | −25.0 | −23.7 | −78.0 | −85.7 |
| years | | (11.86) | (−30.1, −16.3) | (22.3) | (−94.6, −60.0) |
| Enrollment up | 20 | −24.3 | −23.8 | −76.6 | −82.8 |
| to protocol | | (10.55) | (−28.1, −17.1) | (20.8) | (−90.8, −64.6) |
| v3.0 | | | | | |
| Enrollment on | 7 | −26.5 | −25.2 | −88.8 | −88.8 |
| or after | | (6.58) | (−31.9, −22.6) | (8.8) | (−98.5, −81.3) |
| protocol v4.0 | | | | | |

IQR = interquartile range
SD = standard deviation

### Example 6: Upadacitinib Immediate Release Liquid Formulation

For pediatric use, an oral solution was developed to improve acceptability (palatability and swallowability), stability, and manufacturability. Specifically, a stable oral pharmaceutical solution of upadacitinib at 1 mg/mL and 0.5 mg/mL were prepared. Upadacitinib has good solubility at low pH (shown in Table 13). Thus, low pH buffers such as citrate, phosphate, tartrate, and formate are suitable to prepare the oral solution. Buffers with higher pH ranges such as succinate and acetate are also suitable (refer to Example 7) but will result in an oral suspension. Citrate buffer was selected because it has favorable pKa (˜3.1), which is close to the final pH of the oral solution. Accordingly, a formulation was developed based on the solubility of upadacitinib in liquid with citric acid and sodium citrate added to completely dissolve upadacitinib.

Upadacitinib has strong bitterness above concentrations of 0.1 mg/mL. An acceptable and palatable formulation has a bitter intensity below 1.0. Hence, a sweetener, taste masking or modifier agent, and flavoring agent can mitigate the bitter taste of upadacitinib. Sweeteners or combinations of sweeteners such as acesulfame potassium, sodium saccharin, sucralose, neotame, sucrose, maltitol, and xylitol are suitable for this oral solution. Taste modifiers such as sodium chloride, citric acid and monoammonium glycyrrhinizate are also suitable for this oral solution. Flavoring agents such as cherry, orange, bubblegum, strawberry, and mango can enhance the acceptability of the formulation.

Upadacitinib oral solution contains water and sweetener that are potential causes of microbial growth. Upadacitinib oral solution is also a multi-dose formulation. Hence preservative is added into the formulation to prevent microbial proliferation. Preservatives such as sodium benzoate and propyl paraben are suitable based on the final pH of the oral solution. Other preservatives such as sodium metabisulfite, benzoic acid, para-hydroxybenzoate, potassium sorbate and para-hydroxybenzoic acid can also be used based on the final pH of the oral solution or suspension.

The upadacitinib 1 mg/mL oral solution C contained citric acid, sodium citrate, sucralose, sodium benzoate, and water. The 1 mg/mL oral solution C was clear and colorless to light yellow. The composition of certain 1 mg/mL and 0.5 mg/mL oral solutions is shown in Table 14.

TABLE 13
Solubility of upadacitinib in different aqueous media at 37° C.

| | pH | | Solubility |
|---|---|---|---|
| Medium | (Nominal) | pH (Final) | (mg/mL) |
| 0.1N HCl | 1.0 | 2.57 | 38.4 ± 1.5 |
| 50 mM phosphate | 2.01 | 3.08 | 10.5 ± 0.1 |
| 50 mM citrate | 3.00 | 3.39 | 4.48 ± 0.08 |
| 50 mM citrate | 4.01 | 4.16 | 1.00 ± 0.01 |
| 50 mM citrate | 5.03 | 5.01 | 0.289 ± 0.006 |
| 50 mM citrate | 6.01 | 5.96 | 0.196 ± 0.001 |
| 50 mM phosphate | 7.02 | 7.14 | 0.194 ± 0.001 |
| 50 mM phosphate | 8.02 | 7.99 | 0.200 ± 0.013 |
| 50 mM carbonate | 9.02 | 9.11 | 0.199 ± 0.006 |
| Water | 6.02 | 6.92 | 0.240 ± 0.004 |
| Fed State Simulated | 5.01 | 5.10 | 0.455 ± 0.006 |
| Intestinal Fluid (FeSSIF) | | | |
| FeSSIF | 6.50 | 6.58 | 0.262 ± 0.003 |

TABLE 14
Compositions of proposed upadacitinib oral solutions

| | | A | B | C |
|---|---|---|---|---|
| | | 1 | 0.5 | 1 |
| Formulation | | mg/mL | mg/mL | mg/mL |
| Component | Function | Quantity (g) | | |
| upadacitinib freebase | Active | 0.100 | 0.050 | 0.100 |
| sodium benzoate | Preservative | 0.050 | 0.030 | 0.030 |
| citric acid, anhydrous | pH modifier | 0.288 | 0.213 | 0.213 |
| sodium citrate, | Buffer | — | 0.116 | 0.045 |
| dihydrate | | | | |
| sucralose | Sweetener | 0.900 | 0.900 | 0.900 |
| purified water | Solvent | q.s. to 100 | q.s. to 100 | q.s. to 100 |

### Example 7. Upadacitinib Immediate Release or Extended-Release Liquid Formulation (Suspension)

An immediate release oral suspension is prepared to accommodate higher dosage strength or higher pH. The buffers, preservatives, and sweeteners listed in Example 6 can be applied in this formulation. Generally, an extended-release liquid formulation is prepared to provide extended release of upadacitinib for once-daily administration using a release rate modifier, such as an ion exchange resin. The liquid dosage forms comprise an upadacitinib-ion exchange resin complex. The upadacitinib-ion exchange resin complex comprises upadacitinib or a pharmaceutically acceptable salt thereof bound to an ion exchange resin. Suitable ion exchange resins include, but are not limited to, a sulfonated copolymer comprising styrene and divinylbenzene. In some such embodiments, the mobile, or exchangeable, cation is sodium. An exemplary cation ion exchange resin is AmberLite™ IRP 69 (DuPont).

### Example 8: Safety and Efficacy of Upadacitinib for Pediatric Patients with Polyarticular Course Juvenile Idiopathic Arthritis: An Interim Analysis of an Open-Label, Phase 1 Trial (Analysis of Interim Results of Example 1 Through Week 48)

This Example provides additional data through week 48 for the study described in Example 1.

### Objective

The objective of this study was to evaluate the pharmacokinetics, efficacy, and safety of upadacitinib in pediatric patients with polyarticular-course juvenile idiopathic arthritis (pcJIA).

### Method

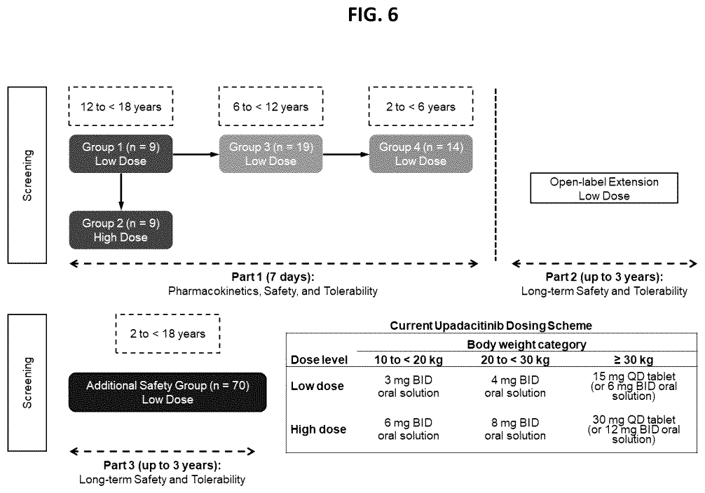

As described above in Example 1, this was an open-label, phase 1 study, enrolling patients aged 2 to <18 years with pcJIA. Patients received bodyweight-based upadacitinib doses using a twice-daily (BID) oral solution or once-daily (QD) extended-release tablet (See Table 15). The study included a 7-day pharmacokinetic assessment, followed by a long-term efficacy and safety evaluation for up to 156 weeks, including an additional long-term safety cohort. This interim analysis includes all available pharmacokinetic and safety data and efficacy data collected through Week 48, excluding patients enrolled in the long-term safety cohort. A schematic depiction of the study design including body weight categories is provided as FIG. 6.

TABLE 15
Upadacitinib Dosing by Body Weight Category
and Dose Level for Subjects 2 to <12 years

| | Body Weight Category* | | |
|---|---|---|---|
| | Current Dosing Scheme Starting with Protocol Version 4.0 | | |
| Dose Level | 10 to <20 kg | 20 to <30 kg | ≥30 kg |
| Low Dose | 3 mg BID | 4 mg BID | 15 mg QD Tablet |
| | Oral Solution | Oral Solution | (Or 6 mg BID Oral |
| | | | Solution if unable |
| | | | to swallow tablet) |
| High Dose | 6 mg BID | 8 mg BID | 30 mg QD Tablet |
| | Oral Solution | Oral Solution | (Or 12 mg BID Oral |
| | | | Solution if unable |
| | | | to swallow tablet) |

| Dose | Previous Dosing Scheme Before Protocol Version 4.0 | | | |
|---|---|---|---|---|
| Level | 10 to <15 kg | 15 to <25 kg | 25 to <40 kg | ≥40 kg |
| Low | 1.6 mg BID IR | 2 mg BID IR | 7.5 mg QD ER | 15 mg QD ER |
| Dose | Oral Solution | Oral Solution | Tablet or | Tablet or |
| | | | 3 mg BID IR | 6 mg BID IR |
| | | | Oral Solution | Oral Solution |
| High | 3.2 mg BID IR | 4 mg BID IR | 15 mg QD ER | 30 mg QD ER |
| Dose | Oral Solution | Oral Solution | Tablet or | Tablet or |
| | | | 6 mg BID IR | 12 mg BID IR |
| | | | Oral Solution | Oral Solution |

BID = twice daily;
QD = once daily
*At least 4 children will be enrolled in each weight category.

### Results

### Patients and Disposition

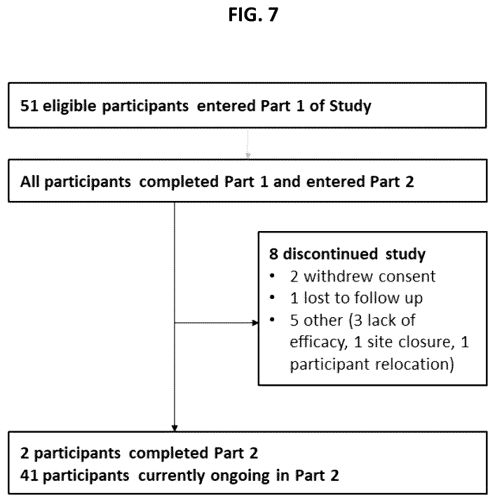

A summary of the patient disposition is provided as FIG. 7. A summary of the patient demographics and disease characteristics is provided in Table 16. With reference to Table 16, the majority of enrolled patients were female (45/57, 78.9%) and had RF-negative polyarticular JIA (42/57, 73.7%). The enrolled patients were between 2 to 17 years old, with a mean disease duration of 3 years. At baseline, 23 (40.4%) patients received methotrexate, 11 (19.3%) patients received oral corticosteroids, and 14 (24.6%) patients reported prior use of bDMARDs.

TABLE 16
Baseline Demographics and Disease Characteristics

| Mean +/− SD (Range) or n (%) | All patients (n = 57) |
|---|---|
| Female, n (%) | 45 (78.9) |
| Age, years, mean ± SD (range) | 9.5 ± 4.41 (2-17) |
| White, n (%) | 55 (96.5) |
| Weight, kg, mean ± SD (range) | 38.05 ± 20.380 (11.0-92.9) |
| pcJIA Type | |
| Extended oligoarticular JIA, n (%) | 7 (12.3) |
| RF-negative polyarticular JIA, n (%) | 42 (73.7) |
| RF-positive polyarticular JIA, n (%) | 7 (12.3) |
| Systemic JIA w active arthritis and w/o | 1 (1.8) |
| active systemic features, n (%) | |
| Duration of pcJIA diagnosis, years, | 2.959 ± 3.3387 (0.05-13.23) |
| mean ± SD (range) | |
| Active joints, mean ± SD (range) | 11.1 ± 7.74 (5-48) |
| C-HAQ | 1.047 ± 0.7746 (0.00-2.88) |
| CRP (mg/L) | 9.11 ± 18.958 (0.2-92.3) |
| ESR (n = 53) | 19.5 ± 19.02 (2-94) |
| Oral corticosteroids, n (%) | 11 (19.3) |
| Methotrexate, n (%) | 23 (40.4) |
| Prior exposure to bDMARDs | 14 (24.6) |

bDMARDs, biological disease-modifying antirheumatic drugs;
CRP, c-reactive protein (normal range ≤2.87 mg/L);
JIA, juvenile idiopathic arthritis;
ESR, erythrocyte sedimentation rate (normal range 3-15 mm/hr);
pcJIA, polyarticular-course juvenile idiopathic arthritis;
n, number of patients;
RF, rheumatoid factor;
SD, standard deviation

### Pharmacokinetics

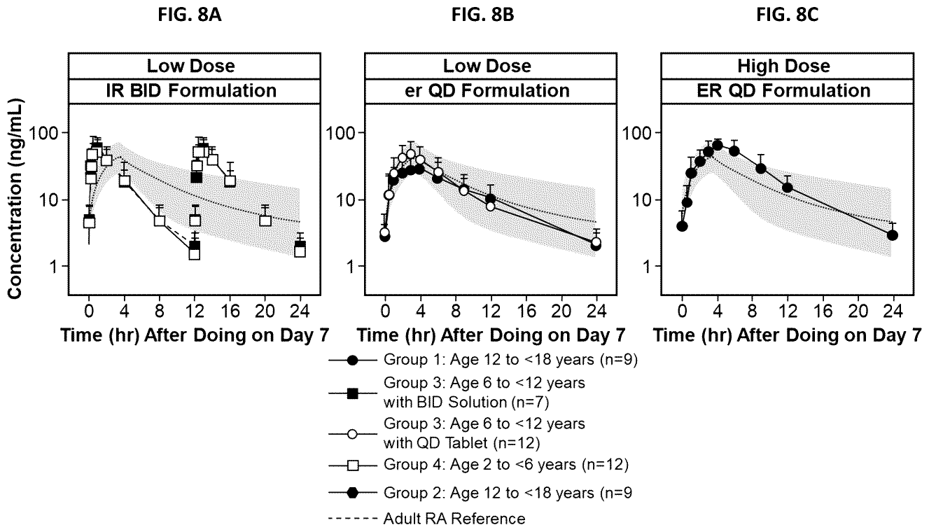

The mean plasma concentration-time profiles by dose level and formulations compared to adult reference profiles simulated based on pharmacokinetic analyses of upadacitinib data in phase 3 RA studies are presented in FIGS. 8A-8C. Adult RA reference represents median (dashed line) and (5^{th}, 95^{th}) percentiles (shaded area) of upadacitinib model-predicted plasma concentrations in adult patients with RA following administration of QD tablets. These reference pharmacokinetic profiles were simulated using a previously developed model for RA patients. The symbols with decreased opacity (over 12-24 hour) for the IR BID regimens are replicated from the observed data (over 0-12 hour) to account for difference in dosing frequency between the BID oral solution and QD tablet formulations.

A summary of the pharmacokinetic parameters of upadacitinib at steady state (i.e., on Day 7) after administration of upadacitinib in patients enrolled in Part 1 is provided in Table 17. During the pharmacokinetic assessment, 13 patients in Group 3 and 12 patients in Group 4 received revised doses, and all other patients received the original doses. Upadacitinib Cₘₐₓ was reached within approximately 3 hours and 1 hour following the administration of the ER tablet and the IR oral solution formulations, respectively. Upadacitinib functional t_{1/2} was approximately 5 hours within a QD dosing interval for the ER tablet and 2 hours within a BID dosing interval for the IR

### Solution

TABLE 17
Geometric Mean (Mean, % CV) Pharmacokinetic Parameters of
Upadacitinib by Study Group and Dosing Regimen (Part 1)

| | | | | | | Age 2 |
|---|---|---|---|---|---|---|
| | Age 12 to <18 years | | | | | to <6 years |
| | Group 1: | Group 2: | Age 6 to <12 years | | | Group 4: |
| | low dose | high dose | Group 3: low dose | | | low dose |
| Pharmacokinetic | QD | QD | QD | BID | | BID |
| parameters | tablet | tablet | tablet | solution | Combined^{g} | solution |
| (units) | (n = 9) | (n = 9) | (n = 12) | (n = 7) | (n = 19) | (n = 12)ⁱ |
| Cₘₐₓ (ng/mL) | 35.1 | 69.8 | 46.9 | 58.9 | 51.0 | 46.6 |
| | (37.2, 35) | (71.2, 19) | (52.3, 46) | (62.5, 35) | (56.1, 41) | (55.8, 56) |
| Tₘₐₓ^{a} (h) | 3.0 | 4.0 | 3.0 | 1.0 | | 1.0 |
| | (1.0-6.0) | (2.0-6.0) | (1.0-6.0)^{f} | (0.5-1.0) | — | (0.5-2.0) |
| Functional t_{1/2}^{b} (h) | 5.53 | 4.79 | 5.20 | 2.39 | — | 2.33 |
| | (1.77)^{e} | (1.12) | (0.949)^{f} | (0.277) | | (0.351)^{j} |
| AUCₜₐᵤ (ng · h/mL) | 269 | 553 | 318 | 198 | — | 184 |
| | (282, 32)^{e} | (572, 26) | (351, 41)^{f} | (209, 36) | | (217, 61)^{j} |
| AUC₀₋₂₄^{c} (ng · h/mL) | 269 | 553 | 318 | 397 | 346 | 369 |
| | (282, 32)^{e} | (572, 26) | (351, 41)^{f} | (417, 36) | (377, 38)^{h} | (433, 61)^{j} |
| CLₛₛ/F (L/h) | 55.7 | 46.5 | 41.6 | 19.7 | — | 15.0 |
| | (58.2, 32)^{e} | (49, 38) | (46.7, 67)^{f} | (20.2, 25) | | (16.8, 49)^{j} |
| CLₛₛ/F_adjusted^{d} (L/h) | 38.1 | 31.8 | 28.5 | 19.7 | 24.7 | 15.0 |
| | (39.8, 32)^{e} | (33.5, 38) | (32.0, 67)^{f} | (20.2, 25) | (27.4, 65)^{h} | (16.8, 49)^{j} |

AUC, area under the plasma concentration-time curve from time 0 to 24 hr (AUC₀₋₂₄) or over a dose interval (AUCₜₐᵤ); BID, twice daily; Cₘₐₓ, maximal plasma concentration; CLₛₛ/F, apparent oral clearance at steady state; CLₛₛ/F_adjusted, CLₛₛ/F adjusted for bioavailability; n, number of patients; QD, once daily; Tₘₐₓ, time to maximal plasma concentration; functional t_{1/2}, functional half-life.
^{a}Median (minimum through maximum).
^{b}Harmonic mean (pseudo-standard deviation).
^{c}For QD tablet, AUC₀₋₂₄ = AUCₜₐᵤ; for BID oral solution, AUC₀₋₂₄ = AUCₜₐᵤ × 2.
^{d}CLₛₛ/F adjusted for the difference in bioavailability between formulations. For QD tablet, CLₛₛ/F_adjusted = 0.684 × CLₛₛ/F; For BID oral solution, CLₛₛ/F_adjusted = CLₛₛ/F.
^{e}n = 8.
^{f}n = 11.
^{g}Cₘₐₓ, AUC₀₋₂₄, and CLₛₛ/F_adjusted of the ER tablet and IR oral solution formulations are combined and summarized together for Group 3.
^{h}n = 18.
ⁱOne patient was excluded for receiving an incorrect dose during the entire Part 1 period, and another patient that did not receive a dose on Day 7 due to an AE of vomiting was also excluded.
^{j}n = 10.

### Efficacy

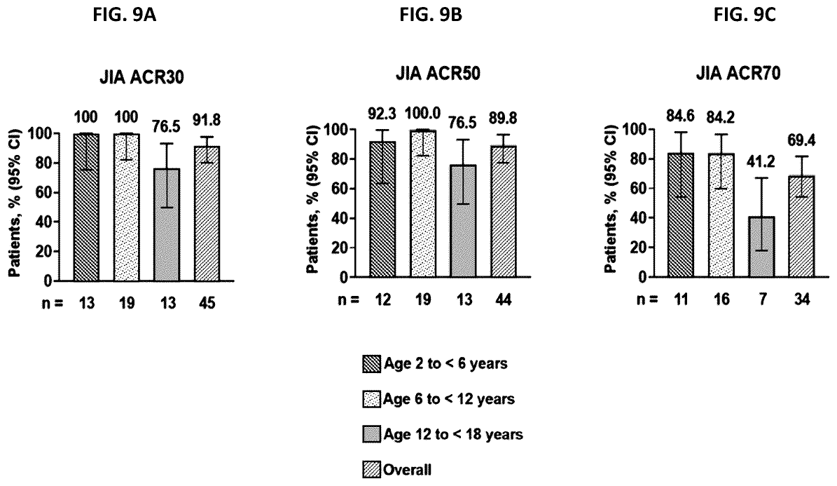

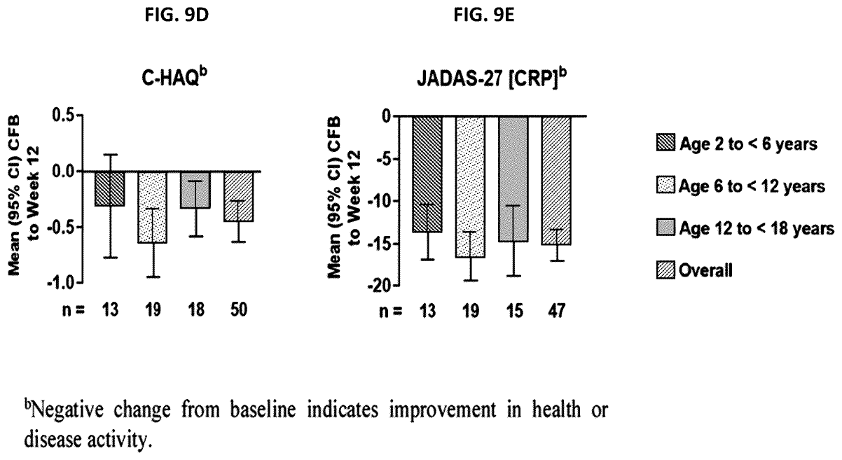

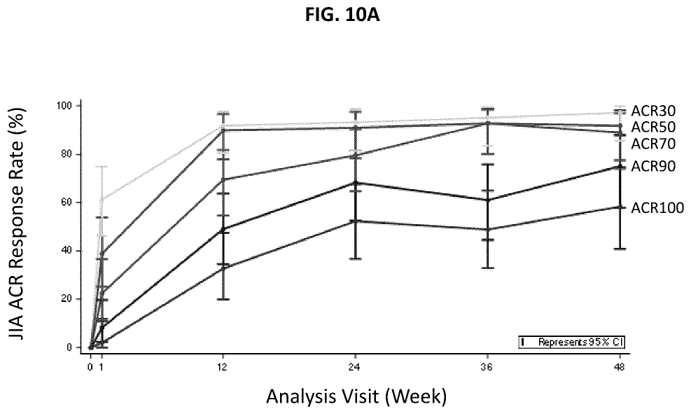

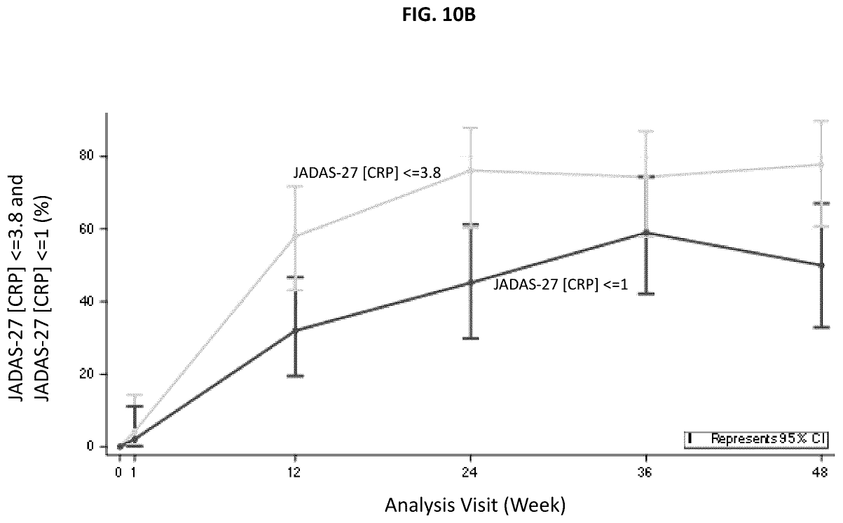

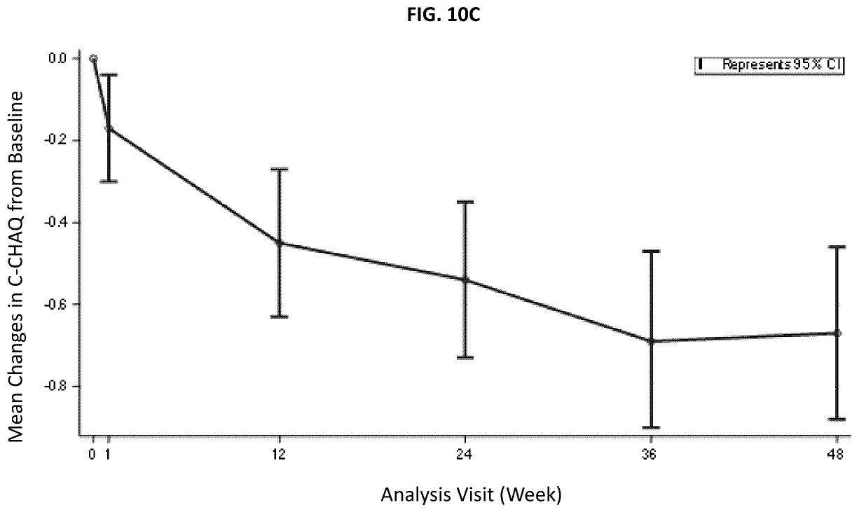

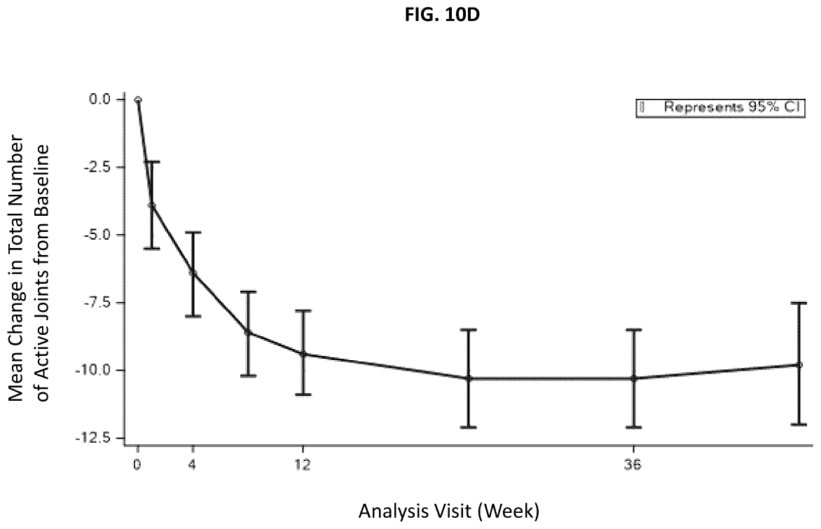

Up to the data cutoff date, 50 of 51 patients from Parts 1 and 2 had efficacy results available at Week 12, and 37 patients had efficacy results available through Week 48, by which point 35 of the 37 patients were being treated with the revised upadacitinib dosing scheme. A summary of the JIA ACR 30/50/70 responses, C-HAQ, and JADAS-27 CRP at Week 12 is presented in FIGS. 9A to 9E, respectively. Overall, the percentage of patients in Parts 1 and 2 who achieved JIA ACR 30/50/70/90/100 response at Week 12 were 91.8/89.8/69.4/49.0/32.7%. The two younger age groups (2 to <6 years and 6 to <12 years) had similar improvements in JIA ACR responses, and the oldest age group (12 to <18 years) had numerically lower rates of JIA ACR responses compared to the younger age groups without statistical significance. Response to upadacitinib was rapid, with 61.2% of patients achieving JIA ACR 30 as early as Week 1. JIA ACR responses continued to improve at Week 24 and were generally maintained through Week 48 (FIG. 10A). Additional key efficacy measurements, including C-HAQ, total number of active joints, Physician's Global Assessment of Disease Activity (VAS), Patient/Parent Global Assessment of Overall Well Being (VAS), and JADAS-27 CRP, JADAS-27 ESR, ESR, and CRP are summarized in Table 18. Across these efficacy measures at Week 12, improvement from baseline was observed in all age groups and changes from baseline were consistent between the age groups. Changes in JADAS-27 ESR were similar to those for JADAS-27 CRP. Twenty-nine (29) of 50 patients (58%) achieved JADAS-27 CRP≤3.8 and 16 of 50 patients (32%) achieved JADAS-27 CRP≤1 at Week 12. The proportions of patients achieving these responses further increased at Week 24 and were generally maintained through Week 48 (FIGS. 10B, 10C, and 10D).

TABLE 18
Summary of Select Efficacy Endpoints at Week 12 (Parts 1 and 2)

| Efficacy Endpoint | | Baseline | Visit | Within Group Change from Baseline | |
|---|---|---|---|---|---|
| Age Group | n | Mean | Mean | Mean (95% CI) | Median (Min, Max) |
| C-HAQ | | | | | |
| 2 to <6 years | 13 | 0.85 | 0.54 | −0.31 (−0.77, 0.15) | −0.38 (−1.3, 1.1) |
| 6 to <12 years | 19 | 1.07 | 0.43 | −0.64 (−0.95, −0.34) | −0.38 (−1.9, 1.1) |
| 12 to <18 years | 18 | 1.04 | 0.71 | −0.33 (−0.58, −0.09) | −0.31 (−1.1, 0.5) |
| Overall | 50 | 1.00 | 0.56 | −0.45 (−0.63, −0.27) | −0.38 (−1.9, 1.1) |

Total Number of Active Joints

| | | | | | |
|---|---|---|---|---|---|
| 2 to <6 years | 13 | 8.1 | 1.1 | −7.0 (−8.8, −5.2) | −6.0 (−12, −3) |
| 6 to <12 years | 19 | 11.6 | 1.4 | −10.2 (−13.0, −7.5) | −8.0 (−24, −4) |
| 12 to <18 years | 18 | 11.7 | 1.5 | −10.2 (−13.3, −7.2) | −9.0 (−25, 0) |
| Overall | 50 | 10.7 | 1.3 | −9.4 (−10.9, −7.8) | −8.0 (−25, 0) |

Physician's Global Assessment of Disease Activity (VAS)

| | | | | | |
|---|---|---|---|---|---|
| 2 to <6 years | 13 | 51.6 | 6.1 | −45.5 (−53.6, −37.5) | −49.0 (−72, −25) |
| 6 to <12 years | 19 | 59.3 | 4.1 | −55.3 (−65.2, −45.3) | −55.0 (−100, −28) |
| 12 to <18 years | 15 | 62.4 | 16.1 | −46.3 (−63.2, −29.4) | −41.0 (−94, 9) |
| Overall | 47 | 58.2 | 8.4 | −49.7 (−56.4, −43.0) | −49.0 (−100, 9) |

Patient/Parent Global Assessment of Overall Well Being (VAS)

| | | | | | |
|---|---|---|---|---|---|
| 2 to <6 years | 13 | 48.4 | 15.6 | −32.8 (−52.2, −13.4) | −38.0 (−81, 37) |
| 6 to <12 years | 19 | 42.3 | 13.5 | −28.8 (−39.2, −18.4) | −29.0 (−61, 9) |
| 12 to <18 years | 18 | 45.8 | 26.2 | −19.6 (−28.3, −10.8) | −23.0 (−45, 8) |
| Overall | 50 | 45.1 | 18.6 | −26.5 (−33.2, −19.8) | −26.0 (−81, 37) |

JADAS27-CRP

| | | | | | |
|---|---|---|---|---|---|
| 2 to <6 years | 13 | 16.81 | 3.17 | −13.65 (−16.88, −10.42) | −12.86 (−22.4, −5.1) |
| 6 to <12 years | 19 | 19.40 | 2.85 | −16.55 (−19.42, −13.68) | −16.90 (−28.9, −7.2) |
| 12 to <18 years | 15 | 20.30 | 5.58 | −14.72 (−18.88, −10.55) | −14.40 (−31.1, −0.3) |
| Overall | 47 | 18.97 | 3.81 | −15.16 (−17.02, −13.30) | −14.59 (−31.1, −0.3) |

JADAS27-ESR

| | | | | | |
|---|---|---|---|---|---|
| 2 to <6 years | 11 | 16.23 | 3.14 | −13.09 (−16.73, −9.46) | −13.70 (−22.4, −5.8) |
| 6 to <12 years | 19 | 19.91 | 2.83 | −17.08 (−20.19, −13.98) | −16.90 (−30.6, −7.2) |
| 12 to <18 years | 15 | 20.41 | 5.78 | −14.63 (−18.86, −10.41) | −13.70 (−31.7, −0.2) |
| Overall | 45 | 19.18 | 3.89 | −15.29 (−17.30, −13.28) | −14.40 (−31.7, −0.2) |

ESR

| | | | | | |
|---|---|---|---|---|---|
| 2 to <6 years | 9 | 25.6 | 11.2 | −14.3 (−25.9, −2.8) | −7.0 (−39, 2) |
| 6 to <12 years | 19 | 18.8 | 8.9 | −9.9 (−17.3, −2.5) | −4.0 (−41, 7) |
| 12 to <18 years | 16 | 13.6 | 13.3 | −0.3 (−5.8, 5.3) | −2.5 (−17, 26) |
| Overall | 44 | 18.3 | 11.0 | −7.3 (−11.7, −2.9) | −4.0 (−41, 26) |

CRP

| | | | | | |
|---|---|---|---|---|---|
| 2 to <6 years | 11 | 19.362 | 0.933 | −18.429 (−36.310, −0.548) | −4.450 (−77.05, 0.06) |
| 6 to <12 years | 18 | 4.573 | 1.312 | −3.261 (−6.110, −0.412) | −0.820 (−16.70, 5.50) |
| 12 to <18 years | 18 | 2.777 | 2.981 | 0.204 (−2.836, 3.245) | −0.150 (−16.49, 17.20) |
| Overall | 47 | 7.346 | 1.862 | −5.484 (−9.981, −0.987) | −0.390 (−77.05, 17.20) |

| | | |
|---|---|---|
| Efficacy Endpoint Age Group | n | Responder n (%), [95% CI] |

JADAS27-CRP ≤3.8 (minimal disease activity)

| | | |
|---|---|---|
| 2 to <6 years | 13 | 9 (69.2), [38.6, 90.9] |
| 6 to <12 years | 19 | 13 (68.4), [43.4, 87.4] |
| 12 to <18 years | 18 | 7 (38.9), [17.3, 64.3] |
| Overall | 50 | 29 (58.0), [43.2, 71.8] |

JADAS27-CRP ≤1 (inactive disease)

| | | |
|---|---|---|
| 2 to <6 years | 13 | 5 (38.5), [13.9, 68.4] |
| 6 to <12 years | 19 | 9 (47.4), [24.4, 71.1] |
| 12 to <18 years | 18 | 2 (11.1), [1.4, 34.7] |
| Overall | 50 | 16 (32.0), [19.5, 46.7] |

### Discussion

In this open-label Phase 1 study, upadacitinib administered as a fixed dose per body weight category was efficacious and well tolerated in pediatric patients with pcJIA. The observed upadacitinib pharmacokinetics in pediatric patients with pcJIA for the QD regimen using the ER formulation and the BID regimen using the IR formulation were consistent with the characterized upadacitinib pharmacokinetics in adults for the respective formulations.

The original dosing regimen of upadacitinib was selected by leveraging pharmacokinetic data of upadacitinib in adult patients with RA and allometric scaling of clearance and volume of distribution based on body weight, with a goal of achieving upadacitinib exposures in patients with pcJIA similar to the exposures which were shown to be optimal in adult RA patients. In this study, a preliminary population pharmacokinetic analysis indicated that the apparent oral clearance of upadacitinib in pediatric patients was underestimated when an estimate of upadacitinib clearance from adult RA patients with a typical exponent of 0.75 was used to describe the relationship between body weight and clearance. Accordingly, a revised dosing regimen was developed and utilized in the younger pediatric patients (the majority of 6 to <12 years and 2 to <6 years) to enable the attainment of upadacitinib exposures comparable to the target exposures in adults with RA. Based on previous analyses, the median (5^{th}, 95^{th} percentile) upadacitinib area under the plasma concentration-time curve (AUC) at steady state in adult patients with RA in phase 3 trials was 358 (234, 701) and 708 (466, 1332) ng·h/mL after daily administration of 15 mg and 30 mg ER tablets, respectively. Compared to the target median adult exposure (AUC₀₋₂₄), the relative median upadacitinib AUC₀₋₂₄ within each group in this study ranged from approximately 0.77 to 1.07 (Table 19). In the younger pediatric patients, where the majority received revised doses, the observed upadacitinib exposures were nearly identical to the target exposures in adult patients with a ratio of 1.01.

In this study, an IR BID oral solution formulation was used for patients with lower body weight (i.e., <30 kg) or unable to swallow tablets. Despite distinct pharmacokinetic profiles due to different dosing frequency, the BID oral solution is expected to provide similar efficacy in pcJIA relative to the QD tablet if similar upadacitinib AUC₀₋₂₄ can be achieved. This is supported by previously conducted exposure-response analyses of key efficacy endpoints in adult RA patients, where a model developed based on data from a BID capsule formulation in phase 2 studies successfully predicted the observed efficacy of the QD tablet formulation in phase 3 studies. The analyses showed that the upadacitinib BID IR and QD ER regimens providing similar AUC₀₋₂₄ were predicted to achieve similar efficacy responses.

Based on the descriptive analyses of efficacy endpoints, improvements in measures of pcJIA disease activity, pain, function, and overall well-being were observed after administration of upadacitinib at Week 12 in this study and were generally maintained through Week 48. Improvements with upadacitinib were observed across the evaluated age groups, which included patients with pcJIA ages 2 to <18 years old, with numerically higher response rates in patients ages 2 to <12 years than those 12 to <18 years. Notably, most patients in the oldest age group had longer disease duration and prior bDMARDs exposure. In comparison, most patients in the younger age groups had shorter disease duration and no prior bDMARD exposure (Table 16). There were no patients with a history of uveitis and no uveitis events occurred during the study. The safety profile of upadacitinib in pediatric patients with pcJIA was generally consistent with the currently known safety profile of upadacitinib in adults and adolescents with inflammatory conditions.

Overall, upadacitinib was well tolerated and efficacious in patients with pcJIA across all age groups (age 2 to <18 years) with plasma exposures comparable to adult RA patients at the evaluated dosing regimens. No new safety risks were observed in pcJIA patients, and the benefit-risk profile of upadacitinib was assessed as favorable based on the safety and efficacy outcomes of the study to date.

TABLE 19
Comparison of Upadacitinib Plasma Exposures between Pediatric
Patients with pcJIA and Adult Patients with RA

| | |
|---|---|
| Median AUC₀₋₂₄ | |
| (ng · h/mL) | Ratio to Adults |

Low-dose level (corresponding to the 15 mg ER tablet in adults)

| | | |
|---|---|---|
| Adult Patients with RA | 358 | |
| Group 1 (12 to <18 years) | 278 | 0.78 |
| Group 3 (6 to <12 years) | 383 | 1.07 |
| Group 4 (2 to <6 years) | 360 | 1.01 |

High-dose level (corresponding to the 30 mg ER tablet in adults)

| | | |
|---|---|---|
| Adult Patients with RA | 708 | |
| Group 2 (12 to <18 years) | 547 | 0.77 |

ER, extended-release;
pcJIA, polyarticular-course juvenile idiopathic arthritis;
RA, rheumatoid arthritis

What is claimed:

1. A method for treating polyarticular course juvenile idiopathic arthritis (pcJIA) in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(ii) administering upadacitinib once daily at a dose of 15 mg (15 mg QD), or
(iv) administering upadacitinib once daily at a dose of 30 mg (30 mg QD).

2. The method of claim 1, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

3. The method of claim 2, wherein the stable oral pharmaceutical solution comprises upadacitinib, a buffer and/or pH adjusting agent, a preservative, a sweetener, and water.

4. The method of claim 2 or 3, wherein the oral solution comprises upadacitinib at a concentration of about 0.5 mg/mL or about 1 mg/mL.

5. The method of any one of claims 1-4, wherein the once daily at a dose of 15 mg of upadacitinib or the once daily at a dose of 30 mg of upadacitinib is administered to the pediatric patient as an extended-release tablet.

6. The method of any one of claims 1-4, wherein the pediatric patient achieves a JIA ACR pediatric 30 response at 12 weeks after the first daily administration.

7. The method of any one of claims 1-4, wherein the pediatric patient achieves a JIA ACR pediatric 50 response at 12 weeks after the first daily administration.

8. The method of any one of claims 1-4, wherein the pediatric patient achieves a JIA ACR pediatric 70 response at 12 weeks after the first daily administration.

9. The method of any one of claims 1-4, wherein the pediatric patient achieves a JIA ACR pediatric 90 response at 12 weeks after the first daily administration.

10. A method for treating systemic juvenile idiopathic arthritis (SJIA) in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

11. The method of claim 10, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

12. The method of claim 11, wherein the stable oral pharmaceutical solution comprises upadacitinib, a buffer and/or pH adjusting agent, a preservative, a sweetener, and water.

13. The method of claim 11 or 12, wherein the oral solution comprises upadacitinib at a concentration of about 0.5 mg/mL or about 1 mg/mL.

14. The method of claim 10, wherein the once daily at a dose of 15 mg of upadacitinib or the once daily at a dose of 30 mg of upadacitinib is administered to the pediatric patient as an extended-release tablet.

15. A method for treating atopic dermatitis (AD) in a pediatric patient less than twelve years old, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

16. The method of claim 15, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

17. The method of claim 16, wherein the stable oral pharmaceutical solution comprises upadacitinib, a buffer and/or pH adjusting agent, a preservative, a sweetener, and water.

18. The method of claim 16 or 17, wherein the oral solution comprises upadacitinib at a concentration of about 0.5 mg/mL or about 1 mg/mL.

19. The method of claim 15, wherein the once daily at a dose of 15 mg of upadacitinib or the once daily at a dose of 30 mg of upadacitinib is administered to the pediatric patient as an extended-release tablet.

20. The method of any one of claims 15-19, wherein the pediatric patient achieves an EASI 75 response at 12 weeks after the first daily administration.

21. The method of any one of claims 15-19, wherein the pediatric patient achieves an EASI 90 response at 12 weeks after the first daily administration.

22. The method of any one of claims 15-19, wherein the pediatric patient achieves an EASI 100 response at 12 weeks after the first daily administration.

23. The method of any one of claims 15-19, wherein the pediatric patient achieves an Investigator's Global Assessment scale for Atopic Dermatitis (vIGA-AD) score of 0 or 1 at 12 weeks after the first daily administration.

24. A method for treating psoriatic arthritis (PsA) in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

25. The method of claim 24, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

26. The method of claim 25, wherein the stable oral pharmaceutical solution comprises upadacitinib, a buffer and/or pH adjusting agent, a preservative, a sweetener, and water.

27. The method of claim 25 or 26, wherein the oral solution comprises upadacitinib at a concentration of about 0.5 mg/mL or about 1 mg/mL.

28. The method of claim 24, wherein the once daily at a dose of 15 mg of upadacitinib or the once daily at a dose of 30 mg of upadacitinib is administered to the pediatric patient as an extended-release tablet.

29. A method for treating ankylosing spondylitis (AS) in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

30. The method of claim 29, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

31. The method of claim 30, wherein the stable oral pharmaceutical solution comprises upadacitinib, a buffer and/or pH adjusting agent, a preservative, a sweetener, and water.

32. The method of claim 30 or 31, wherein the oral solution comprises upadacitinib at a concentration of about 0.5 mg/mL or about 1 mg/mL.

33. The method of claim 29, wherein the once daily at a dose of 15 mg of upadacitinib or the once daily at a dose of 30 mg of upadacitinib is administered to the pediatric patient as an extended-release tablet.

34. A method for treating non-radiographic axial spondyloarthritis (nr-axSpA) in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

35. The method of claim 34, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

36. The method of claim 35, wherein the stable oral pharmaceutical solution comprises upadacitinib, a buffer and/or pH adjusting agent, a preservative, a sweetener, and water.

37. The method of claim 35 or 36, wherein the oral solution comprises upadacitinib at a concentration of about 0.5 mg/mL or about 1 mg/mL.

38. The method of claim 34, wherein the once daily at a dose of 15 mg of upadacitinib or the once daily at a dose of 30 mg of upadacitinib is administered to the pediatric patient as an extended-release tablet.

39. A method for treating hidradenitis suppurative in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

40. The method of claim 39, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

41. The method of claim 40, wherein the stable oral pharmaceutical solution comprises upadacitinib, a buffer and/or pH adjusting agent, a preservative, a sweetener, and water.

42. The method of claim 40 or 41, wherein the oral solution comprises upadacitinib at a concentration of about 0.5 mg/mL or about 1 mg/mL.

43. The method of claim 39, wherein the once daily at a dose of 15 mg of upadacitinib or the once daily at a dose of 30 mg of upadacitinib is administered to the pediatric patient as an extended-release tablet.

44. A method for treating systemic lupus erythematosus in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

45. The method of claim 44, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 8 mg of upadacitinib is administered to the pediatric patient as a stable oral pharmaceutical solution.

46. The method of claim 45, wherein the stable oral pharmaceutical solution comprises upadacitinib, a buffer and/or pH adjusting agent, a preservative, a sweetener, and water.

47. The method of claim 45 or 46, wherein the oral solution comprises upadacitinib at a concentration of about 0.5 mg/mL or about 1 mg/mL.

48. The method of claim 44, wherein the once daily at a dose of 15 mg of upadacitinib or the once daily at a dose of 30 mg of upadacitinib is administered to the pediatric patient as an extended-release tablet.

49. A method for treating ulcerative colitis in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(ii) administering upadacitinib once daily at a dose of 15 mg (15 mg QD), or
(iv) administering upadacitinib once daily at a dose of 30 mg (30 mg QD).

50. The method of claim 49, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

51. The method of claim 50, wherein the stable oral pharmaceutical solution comprises upadacitinib, a buffer and/or pH adjusting agent, a preservative, a sweetener, and water.

52. The method of claim 50 or 51, wherein the oral solution comprises upadacitinib at a concentration of about 0.5 mg/mL or about 1 mg/mL.

53. The method of claim 49, wherein the once daily at a dose of 15 mg of upadacitinib or the once daily at a dose of 30 mg of upadacitinib is administered to the pediatric patient as an extended-release tablet.

54. A method for treating Crohn's disease in a pediatric patient, the method comprising administering a therapeutically effective amount of upadacitinib to the pediatric patient, wherein
if the pediatric patient has a body weight in a range from about 10 kg to less than about 20 kg:
(i) administering upadacitinib twice daily at a dose of 3 mg each (3 mg BID), or
(ii) administering upadacitinib twice daily at a dose of 6 mg each (6 mg BID);
if the pediatric patient has a body weight in a range from about 20 kg to less than about 30 kg:
(i) administering upadacitinib twice daily at a dose of 4 mg each (4 mg BID), or
(ii) administering upadacitinib twice daily at a dose of 8 mg each (8 mg BID); and
if the pediatric patient has a body weight of about 30 kg or greater:
(i) administering upadacitinib twice daily at a dose of 6 mg each (6 mg BID),
(ii) administering upadacitinib twice daily at a dose of 12 mg each (12 mg BID),
(iii) administering upadacitinib once daily at a dose of 15 mg (15 mg QD), or
(iv) administering upadacitinib once daily at a dose of 30 mg (30 mg QD).

55. The method of claim 54, wherein the twice daily at a dose of 3 mg of upadacitinib, the twice daily at a dose of 4 mg of upadacitinib, the twice daily at a dose of 6 mg of upadacitinib, the twice daily at a dose of 8 mg of upadacitinib, or the twice daily at a dose of 12 mg of upadacitinib is administered to the pediatric patient as a stable oral pharmaceutical solution.

56. The method of claim 55, wherein the stable oral pharmaceutical solution comprises upadacitinib, a buffer and/or pH adjusting agent, a preservative, a sweetener, and water.

57. The method of claim 55 or 56, wherein the oral solution comprises upadacitinib at a concentration of about 0.5 mg/mL or about 1 mg/mL.

58. The method of claim 54, wherein the once daily at a dose of 15 mg of upadacitinib or the once daily at a dose of 30 mg of upadacitinib is administered to the pediatric patient as an extended-release tablet.

59. A stable oral pharmaceutical solution comprising upadacitinib or a pharmaceutically acceptable salt or solid-state form thereof, a buffer and/or pH adjusting agent, a preservative, a sweetener and water.

60. The stable oral pharmaceutical solution of claim 59, comprising the anhydrous free base upadacitinib at a concentration of about 0.5 mg/mL.

61. The stable oral pharmaceutical solution of claim 59, comprising the anhydrous free base upadacitinib at a concentration of about 1 mg/mL.

62. The stable oral pharmaceutical solution of claim 59, wherein the buffer is selected from the group consisting of citrate, phosphate, tartrate, succinate, formate, acetate, and combinations thereof.

63. The stable oral pharmaceutical solution of claim 59, wherein the pH adjusting agent is selected from the group consisting of citric acid, phosphoric acid, tartaric acid, succinic acid, formic acid, acetic acid, and combinations thereof.

64. The stable oral pharmaceutical solution of claim 59, wherein the preservative is selected from the group consisting of sodium benzoate, benzoic acid, propyl paraben, sodium metabisulfite, potassium sorbate, hydroxyparabenzoic acid, hydroxyparabenzoate, and combinations thereof.

65. The stable oral pharmaceutical solution of claim 59, wherein the sweetener selected from the group consisting of sucralose, acesulfame potassium, sodium saccharin, neotame, sucrose, maltitol, xylitol, and combinations thereof.

66. The stable oral pharmaceutical solution of claim 59, comprising anhydrous free base upadacitinib, citric acid, sodium citrate, sodium benzoate, sucralose, and water.

67. The stable oral pharmaceutical solution of claim 59, wherein the stable oral pharmaceutical solution has a pH in a range from about 2 to about 5.

68. The stable oral pharmaceutical solution of claim 59, wherein the stable oral pharmaceutical solution has a pH in a range from about 3 to about 4.

69. The stable oral pharmaceutical solution of claim 59, wherein the stable oral pharmaceutical solution has a pH in a range from about 2.5 to about 3.5.

70. The method of any one of claims 1-4, wherein the pediatric patient achieves a JIA ACR pediatric 30 response at 24 weeks after the first daily administration.

71. The method of any one of claims 1-4, wherein the pediatric patient achieves a JIA ACR pediatric 50 response at 24 weeks after the first daily administration.

72. The method of any one of claims 1-4, wherein the pediatric patient achieves a JIA ACR pediatric 70 response at 24 weeks after the first daily administration.

73. The method of any one of claims 1-4, wherein the pediatric patient achieves a JIA ACR pediatric 90 response at 24 weeks after the first daily administration.

74. The method of any one of claims 1-4, wherein the pediatric patient achieves a JIA ACR pediatric 30 response at 48 weeks after the first daily administration.

75. The method of any one of claims 1-4, wherein the pediatric patient achieves a JIA ACR pediatric 50 response at 48 weeks after the first daily administration.

76. The method of any one of claims 1-4, wherein the pediatric patient achieves a JIA ACR pediatric 70 response at 48 weeks after the first daily administration.

77. The method of any one of claims 1-4, wherein the pediatric patient achieves a JIA ACR pediatric 90 response at 48 weeks after the first daily administration.
